(19) United States
(12) Patent Application Publication
(10) Pub. No.: US 2007/0087985 A1
(43) Pub. Date: Apr. 19, 2007

(54) MRCK-related compositions and methods

(76) Inventors: Gregg G. Gundersen, Tenafly, NJ (US); Edgar Rodrigues Gomes, New York, NY (US)

Correspondence Address: COOPER & DUNHAM, LLP, 1185 AVENUE OF THE AMERICAS, NEW YORK, NY, 10036, US

(21) Appl. No.: 11/405,735

(22) Filed: Apr. 18, 2006

Related U.S. Application Data
(60) Provisional application No. 60/673,193, filed on Apr. 19, 2005.

(51) Int. Cl. A61K 48/00; C12Q 1/68; C07H 21/02

(52) U.S. Cl. 514/44; 435/6; 536/23.1

(57) ABSTRACT

This invention provides a composition comprising a pharmaceutically acceptable carrier and a nucleic acid which, when introduced into a cell wherein MRCK is expressed, inhibits the expression of MRCK in the cell. This invention further provides a method for identifying an agent that inhibits MRCK expression in a cell. This invention further provides a method for identifying an agent that inhibits MRCK-mediated kinase activity in a cell. This invention further provides a method for inhibiting the MRCK-mediated migration of a cell in a subject. Finally, this invention provides related methods of treating a subject.

This application claims the benefit of U.S. Provisional Application No. 60/673,193, filed Apr. 19, 2005, the contents of which are incorporated herein by reference into the subject application.

The invention disclosed herein was made with government support from Grant No. GM 062938 from the National Institutes of Health. Accordingly, the U.S. Government has certain rights in this invention.

Throughout this application, various publications are referred to by Arabic numerals within parentheses. Full citations for these publications are presented immediately before the claims. Disclosures of these publications in their entireties are hereby incorporated by reference into this application in order to more fully describe the state of the art to which this invention pertains.

### BACKGROUND OF THE INVENTION

Directional cell migration is essential for development, wound healing, and immune function. In migrating fibroblasts, endothelial cells, astrocytes and neurons, the microtubule (MT) organizing center (MTOC) is reoriented to a position between the leading edge and the nucleus (12, 15, 18, 19, 20, 26, 39). T cells also reorient their MTOC toward the site of interaction with target cells (27). As the Golgi colocalizes with the MTOC, the reorientation of the MTOC gives the cell an overall polarity that is thought to contribute to polarized delivery of membrane precursors and perhaps actin regulatory factors towards the leading edge (3, 41).

An important unsolved question is how the cell uses spatial cues, signalling and the cytoskeleton to reorient the MTOC to a specific location. The small GTPase Cdc42 is a key regulator of MTOC reorientation in a number of systems. Cdc42 was initially implicated in MTOC reorientation in T cells (53). Cdc42 is also involved in Golgi and MTOC reorientation in wound edge fibroblasts (36, 39), astrocytes (12) and shear-stressed endothelial cells (56). Cdc42 acts through a Par6-atypical PKCξ complex in astrocytes and endothelial cells (12, 56). Cytoplasmic dynein and its regulator, dynactin, are also involved in MTOC reorientation (12, 39) and both components localize to the leading edge of migrating cells where they colocalize with MT ends (11).

In many systems, dynein and dynactin regulate the position of the MTOC and the nucleus (10, 34). For example, in Aspergillus nidulans, they position nuclei along the germ tube and with dynein (or dynactin) mutations, nuclei remain at the base of the tube (34, 60). In budding yeast, dynein and dynactin contribute to positioning the nucleus in the bud neck and moving the nucleus into the bud, probably by pulling on cortical MTs (1, 7). In early C. elegans and Xenopus embryos, dynein and dynactin contribute to the movements of the pronucleus and MT asters (17, 49). Nuclear migration to the cortex of Drosophila syncytial blastoderms is dynein dependent (42). Dynein and its regulator, Lis1, have also been implicated in maintaining spindle position in epithelia (6, 16), fibroblasts (37) and in coordinating nuclear and centrosome movements in migrating neurons (48, 51, 55). How dynein works in each of these systems is unclear. Studies of asymmetric division implicate Cdc42, Par6-atypical PKC, other Par proteins, and heterotrimeric G proteins in regulating pulling forces that may be due to dynein (2).

### SUMMARY OF THE INVENTION

This invention provides a composition comprising a pharmaceutically acceptable carrier and a nucleic acid which, when introduced into a cell wherein MRCK is expressed, inhibits the expression of MRCK in the cell.

This invention further provides a method for identifying an agent that inhibits MRCK expression in a cell comprising (a) contacting the cell with the agent under conditions which would permit the cell to express MRCK in the absence of the agent, (b) after a suitable period of time, determining the level of MRCK expression in the cell; and (c) comparing the level of MRCK expression determined in step (b) with the level of MRCK expression determined in the cell in the absence of the agent, whereby a lower amount of expression in the presence of the agent indicates that the agent inhibits MRCK expression in the cell.

This invention further provides a method for identifying an agent that inhibits MRCK-mediated kinase activity in a cell comprising (a) contacting the cell with the agent under conditions which would permit the cell to express MRCK in the absence of the agent, (b) after a suitable period of time, determining the level of MRCK-mediated kinase activity in the cell and (c) comparing the level of MRCK-mediated kinase activity determined in step (b) with the MRCK-mediated kinase activity determined in the cell in the absence of the agent, whereby a lower amount of activity in the presence of the agent indicates that the agent inhibits MRCK-mediated kinase activity in the cell.

This invention further provides a method for inhibiting the MRCK-mediated migration of a cell in a subject comprising administering to the subject a therapeutically effective amount of an agent that inhibits MRCK expression in the cell.

This invention further provides a method for treating a subject afflicted with a condition characterized by metastasizing cells comprising administering to the subject a therapeutically effective amount of an agent that inhibits MRCK expression, thereby treating the subject.

This invention further provides a method for inhibiting scar formation in a subject following the infliction of physical trauma comprising administering to the subject a prophylactically effective amount of an agent that inhibits MRCK expression, thereby inhibiting scar formation in the subject.

Finally, this invention provides a method for reducing inflammation in a subject comprising administering to the subject a therapeutically effective amount of an agent that inhibits MRCK expression, thereby reducing inflammation in the subject.

### BRIEF DESCRIPTION OF THE FIGURES

FIGS. 1A-1I

Rearward nuclear movement reorients the MTOC. A, C. Frames from a timelapse recording of a starved wound edge 3T3-GFPTub cell before (A) and after (C) LPA treatment (time is in hr:min). LPA (2 μM) was added at 26 min. The wound edge is at the top of the panels. The MTOC is the bright dot at the focus of the MT array, while the nucleus appears as a dark area outlined by GFP tubulin fluorescence. B, D. Traces of the MTOC (blue) and nucleus centroid (red) positions before (B) and after (D) addition of 2 μM LPA. E. Superimposition of the cell outline, nucleus and MTOC from frame 0:31 (orange; beginning of nuclear movement) and frame 1:20 (blue; end of nuclear movement) shows rearward movement of the nucleus relative to the leading edge (arrows). F. Average positions of the nucleus (red) and MTOC (blue) relative to the cell centroid (defined as “0”) in fixed populations of starved wound-edge 3T3 cells that were untreated (−LPA) or treated with LPA (+LPA) for the indicated times. Positive values are toward the leading edge; negative values toward the rear of the cell. G. Representative images of wounded serum-starved 3T3 cells treated with or without LPA for 2 hr. Fixed cells were stained for Tyr tubulin (green), pericentrin (red, but appears yellow due to overlap with tubulin staining) and nuclei with DAPI (blue). H. Frames from a two channel recording (phase contrast and fluorescence) of wounded 3T3-GFPTub monolayers treated with 1% serum (at time 0:00 in hr:min). Phase images are shown with the position of the MTOC determined from fluorescence images indicated by a red triangle. I. Kymograph of fluorescence images corresponding to the box in H. The MTOC remains stationary while the nucleus moves rearward; MTOC reorientation occured at 1:31 (asterisk). Bars in A, G, H: 10 μm.

FIGS. 2A-2G

Actin retrograde flow is involved in nuclear movement. A. Region of 3T3-GFPTub cell used for kymograph in B. B. Kymograph showing retrograde movement of MTs and nucleus before and after 2 μM LPA addition. Arrowheads indicate MTs that exhibit retrograde movement in the lamella after LPA addition. The nucleus is the broad dark band at the bottom of the kymograph and begins moving rearward in concert with MTs. C, D. Wound edge starved NIH-3T3 cells were microinjected with R-actin and time lapse images of actin speckles were acquired before (C) or after (D) addition of 2 μM LPA. Kymographs of a region of the lamella are shown (separate cells are shown in C and D). Actin speckles (arrowheads) remain stationary in the absence of LPA (C) but move away from the leading edge upon LPA addition (D). Note that the nucleus remains stationary in the absence of LPA (C) but moves rearward at the same rate as actin speckles after LPA addition (D). E. Starved wounded monolayers of NIH-3T3 cells were either left untreated or treated with 2 μM LPA with or without BB or CD as indicated. After 2 hr, coverslips were fixed, stained and the extent of MTOC reorientation determined. The level of random “reorientation” is ˜33%. F. The average positions of the MTOC and nucleus from the cells treated as in E were determined and plotted. G. Actin distribution (phallodin staining) in wound edge NIH-3T3 cells treated as indicated. Bar, G: 10 μm.

FIGS. 3A-3I

Cdc42 and MRCK activate nuclear movement to reorient the MTOC. A. Starved wound edge NIH-3T3 cells were injected with L61Cdc42, N17Cdc42 or Pak-CRIB domain proteins and then treated with 2 μM LPA for 2 hr. Cells were then fixed, stained and the positions of the nucleus and MTOC were determined. MTOC reorientation is indicated as YES (>60%); NO (<40%). B. Starved wound edge NIH-3T3 cells were injected with DNA encoding myc-hPAK1 (hPAK1(83-149)), HA-MRCK-TM or Flag-MRCK-CPC. After expression (2 hr) cells were treated with 2 μM LPA for 2 hr, fixed, stained and the extent of MTOC reorientation determined. C. Serum starved wound edge NIH-3T3 cells were injected with DNA encoding wild-type HA-L61Cdc42, HA-L61Cdc42 plus flag-MRCK-CPC or flag-MRCK-WT (MRCK-WT). After expression (3 hr), cells were fixed, stained and the extent of MTOC reorientation was determined. D, E. Average nucleus and MTOC positions of cells from B and C were determined and plotted. F. Representative images of cells microinjected with the indicated constructs and treated with LPA as indicated before fixing and staining. Arrows indicate cells with expressed protein (green), pericentrin and β-catenin (red) and DNA (blue). G. Actin (phalloidin staining) in a cell injected with MRCK-TM (arrow) and treated with LPA. H. Starved wound edge NIH-3T3 cells were injected with DNA encoding GFP, flag-MRCK-WT or HA-MRCK-TM and treated with serum for 12 h to stimulate migration. Cells were fixed and stained and the percentage of expressing cells remaining at the wound edge determined. I. Representative images of serum-stimulated cells expressing MRCK-TM and MRCK WT as in H. The expressing cells are outlined and the wound edge is indicated by dotted line. MTs are shown in green, nuclei in blue; the MTOC (at the focus of the MT array) is indicated by a yellow triangle. Bars in F, G, I: 10 μm.

FIGS. 4A-4E

LPA, Cdc42 and MRCK induce activation of myosin II. A. Western blot of total cell extracts of starved cells treated with 2 uM LPA (+LPA) or left untreated (−LPA), and blotted for pSer19 MLC and a-tubulin (Tub) as a loading control. B, D. Starved wound edge cell microinjected with (B) HA-L61Cdc42 DNA (arrow) or (D) flag-MRCK-WT or flag-MRCK-CPC (arrows) and 2.5 hr later fixed and stained for pSer19 MLC. C, E. Quantification of pSer19-MLC fluorescence intensity in cells expressing (C) HA-L61 Ccd42 or (E) MRCK constructs. Bars in B. D: 10 μm.

FIGS. 5A-5E

Dynein is not involved in nuclear movement but is necessary to maintain the MTOC at the cell centroid. A. Frames from a time-lapse recording of a starved wound edge 3T3-GFPTub cell microinjected with DIC mAb before adding 2 μM LPA (5 min before image acquisition; time in hr:min). The wound edge is at the top of the image. After the recording, cells were fixed and stained for mouse IgG to confirm microinjection (inset). Boxed region in the first panel was used for the kymograph in C. B. Traces of the MTOC (blue) and nucleus centroid (red) positions from the time-lapse recording. C. Kymograph of the region in A. Arrowheads indicate MTs in the lamella moving rearward at the same speed as the nucleus (appears as a dark band). D. MTOC reorientation of starved wound edge NIH-3T3 cells microinjected with DIC mAb and treated with 2 μM LPA (2 hr), or cells injected with flag-MRCK-WT DNA and after expression (2 hr), injected with either DIC mAb or human IgG (HuIgG). Cells were fixed, stained and the extent of MTOC reorientation was determined. E. Average positions of the nucleus and MTOC from the cell centroid for cells treated as in D. Bar in A: 10 μm.

FIGS. 6A-6F

Par6, PKCξ and MT dynamics are not involved nuclear movement but are necessary to maintain the MTOC at the cell centroid. A. Starved wound edge NIH-3T3 cells were injected with DNA encoding full-length Flag-Par-6 or HA-kdPKCξ. After expression (2 hr) cells were treated with 2 UM LPA (2 hr), fixed, stained and the extent of MTOC reorientation determined. B. Average positions of the nucleus and MTOC relative to the cell centroid for cells treated as in A. C. Representative fields of LPA-treated cells expressing Flag-Par-6 and HA-kdPKCξ (arrows) stained for MTs (green), pericentrin (red), DNA (blue) and tags (insets). D. Starved wound edge NIH-3T3 cells treated with 2 μM LPA alone or with 100 nM nocodazole (Nz). After 2 h, some cells were fixed or Nz was washed out and replaced with medium containing LPA for another 2h (LPA+Nz wash). Before Nz washout, some cells were microinjected with DIC mAb (LPA+DIC mAb+Nz wash) or HuIgG (LPA+HuIgG+Nz wash). Cells were fixed, stained and the extent of MTOC reorientation determined. E. Average positions of the nucleus and MTOC relative to the cell centroid in the cells treated as in D. F. Representative fields of LPA-stimulated cells treated as indicated (Nz was at 100 nM). Cells were stained as in C (insets show injected Ab). Bars in C, F: 10 μm.

FIG. 7

Two Cdc42 regulated pathways lead to MTOC reorientation. LPA activates Cdc42 to regulate separate actin and MT dependent pathways that result in MTOC reorientation. Cell diagrams (nucleus blue, MTOC red) show phenotypes resulting from inhibition of each pathway. Dashed line represents a horizontal line through the cell center. MRCK is sufficient to stimulate MTOC reorientation and so may also regulate the Par6-PKCξ-dynein-dynactin pathway (dotted arrows).

FIG. 8

Human MRCK alpha cDNA sequence (SEQ. ID. NO:1)

FIG. 9

Human MRCK alpha protein sequence (SEQ. ID. NO:2)

FIG. 10

Human MRCK beta cDNA sequence (SEQ. ID. NO:3)

FIG. 11

Human MRCK beta protein sequence (SEQ. ID. NO:4)

FIG. 12

Human MRCK gamma cDNA sequence (SEQ. ID. NO:5)

FIG. 13

Human MRCK gamma protein sequence (SEQ. ID. NO:6)

### DETAILED DESCRIPTION OF THE INVENTION

Terms

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“Agent” shall mean any chemical entity, including, without limitation, a glycomer, a protein, an antibody, a lectin, a nucleic acid, a small molecule, and any combination thereof. Examples of possible agents include, but are not limited to, a ribozyme, a DNAzyme and an siRNA molecule.

“Cancer” includes, without limitation, biliary tract cancer; brain cancer, including glioblastomas and medulloblastomas; breast cancer; cervical cancer; choriocarcinoma; colon cancer; endometrial cancer; esophageal cancer; gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer; lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; colorectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor.

“DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

“Expression” of MRCK includes, without limitation, (i) transcription of MRCK-encoding DNA and (ii) translation of MRCK-encoding mRNA. Thus, detecting the “level” of MRCK expression may include for example, detecting the level of MRCK-encoding mRNA and/or the level of MRCK itself.

“MRCK” shall mean myotonic dystrophy kinase-related Cdc42-binding kinase. MRCK can be, for example, from human or any other species which produces this protein. Nucleotide and amino acid sequences for MRCKs are known (e.g., Genbank Nos. NM_{—}003607 and NM_{—}014826 (nucleotide) and Nos. NP_{—}003598 and NP_{—}055641 (amino acid) for MRCK alpha, Genbank Nos. NM_{—}006035 (nucleotide) and NP_{—}006026 (amino acid) for MRCK beta and Genbank Nos. NM_{—}017525 (nucleotide) and NP_{—}059995 (amino acid) for MRCK gamma).

“MRCK-mediated kinase activity” shall include, for example, phosphorylation of the myosin light chain (MLC) (28) and the myosin binding subunit of myosin phosphatase (54, 62), phosphorylation of Ezrin, Radixin and Moesin (ERM) (35) and phosphorylation of LIM kinase 1 and LIM kinase 2 (63).

“Nucleic acid” shall mean any nucleic acid molecule, including, without limitation, DNA (e.g., cDNA), RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Physical trauma” is any incident which results in harm to bodily tissue, and includes, for example, a heat-caused burn, an injury caused by a blunt object, a laceration and a chemical burn. Physical trauma can be incurred, for example, during the course of surgery or other medical procedures, and as the result of an accident.

“Polypeptide” and “protein” are used interchangeably herein, and each means a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation.

“Ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

“siRNA” shall mean small interfering ribonucleic acid. Methods of designing and producing siRNA to decrease the expression of a target protein are well known in the art.

“Subject” shall mean any organism including, without limitation, a mammal such as a mouse, a rat, a dog, a guinea pig, a ferret, a rabbit and a primate. In the preferred embodiment, the subject is a human being.

“Therapeutically effective amount” of an agent means an amount of the agent sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art. In one embodiment, the therapeutically effective amount is from about 1 mg of agent/subject to about 1 g of agent/subject per dosing. In another embodiment, the therapeutically effective amount is from about 10 mg of agent/subject to 500 mg of agent/subject. In a further embodiment, the therapeutically effective amount is from about 50 mg of agent/subject to 200 mg of agent/subject. In a further embodiment, the therapeutically effective amount is about 100 mg of agent/subject. In still a further embodiment, the therapeutically effective amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject.

“Treating” a disorder shall mean slowing, stopping or reversing the disorder's progression. In the preferred embodiment, treating a disorder means reversing the disorder's progression, ideally to the point of eliminating the disorder itself.

### EMBODIMENTS OF THE INVENTION

This invention provides a composition comprising a pharmaceutically acceptable carrier and a nucleic acid which, when introduced into a cell wherein MRCK is expressed, inhibits the expression of MRCK in the cell. In one embodiment, the nucleic acid is selected from the group consisting of siRNA, an anti-sense molecule a DNAzyme and a ribozyme. In another embodiment, the MRCK is human MRCK. In another embodiment, the MRCK is encoded by the nucleic acid sequence set forth in SEQ. ID. NO:1.

This invention further provides a method for identifying an agent that inhibits MRCK expression in a cell comprising (a) contacting the cell with the agent under conditions which would permit the cell to express MRCK in the absence of the agent, (b) after a suitable period of time, determining the level of MRCK expression in the cell and (c) comparing the level of MRCK expression determined in step (b) with the level of MRCK expression determined in the cell in the absence of the agent, whereby a lower amount of expression in the presence of the agent indicates that the agent inhibits MRCK expression in the cell. In one embodiment, the agent is a nucleic acid. In another embodiment, the agent is a polypeptide. In another embodiment, the MRCK is human MRCK. In another embodiment, the MRCK is encoded by the nucleic acid sequence set forth in SEQ. ID. NO:1.

This invention further provides a method for identifying an agent that inhibits MRCK-mediated kinase activity in a cell comprising (a) contacting the cell with the agent under conditions which would permit the cell to express MRCK in the absence of the agent, (b) after a suitable period of time, determining the level of MRCK-mediated kinase activity in the cell and (c) comparing the level of MRCK-mediated kinase activity determined in step (b) with the MRCK-mediated kinase activity determined in the cell in the absence of the agent, whereby a lower amount of activity in the presence of the agent indicates that the agent inhibits MRCK-mediated kinase activity in the cell. In one embodiment, the agent is a nucleic acid. In another embodiment, the agent is a polypeptide. In another embodiment, the MRCK is human MRCK. In another embodiment, the MRCK is encoded by the nucleic acid sequence set forth in SEQ. ID. NO:1.

This invention further provides a method for inhibiting the MRCK-mediated migration of a cell in a subject comprising administering to the subject a therapeutically effective amount of an agent that inhibits MRCK expression in the cell. In one embodiment, the cell is selected from the group consisting of a fibroblast, a skin cell, an immune cell and a metastasizing cell. In another embodiment, the immune cell is selected from the group consisting of a macrophage, a lymphocyte, a T-cell and a neutrophil. In another embodiment, the agent is selected from the group consisting of an siRNA molecule, an anti-sense molecule, a DNAzyme and a ribozyme.

This invention further provides a method for treating a subject afflicted with a condition characterized by metastasizing cells comprising administering to the subject a therapeutically effective amount of an agent that inhibits MRCK expression, thereby treating the subject. In one embodiment, the subject is human. In another embodiment, the condition is cancer. In another embodiment, the agent is selected from the group consisting of an siRNA molecule, an anti-sense molecule, a DNAzyme and a ribozyme

This invention also provides a method for inhibiting scar formation in a subject following the infliction of physical trauma comprising administering to the subject a prophylactically effective amount of an agent that inhibits MRCK expression, thereby inhibiting scar formation in the subject. In one embodiment, the subject is human. In another embodiment, the agent is selected from the group consisting of an siRNA molecule, an anti-sense molecule, a DNAzyme and a ribozyme. The agent can be administered following, and where applicable, before and during the event causing the trauma. For example, if the event is a surgical procedure on vascular tissue, the agent can be administered intravenously before, during and after the surgery. If the event is a chemical burn, the agent can be topically administered after the event.

Finally, this invention provides a method for reducing inflammation in a subject comprising administering to the subject a therapeutically effective amount of an agent that inhibits MRCK expression, thereby reducing inflammation in the subject. In one embodiment, the subject is human. In another embodiment, the agent is selected from the group consisting of an siRNA molecule, an anti-sense molecule, a DNAzyme and a ribozyme.

This invention will be better understood from the Experimental Details which follow. However, one skilled in the art will readily appreciate that the specific methods and results discussed are merely illustrative of the invention as described more fully in the claims which follow thereafter.

Experimental Details

Synopsis

While Cdc42, Par6-PKCξ, dynein and dynactin have also been implicated in MTOC reorientation in migrating cells, whether Cdc42 regulates dynein-dependent pulling of MTs or other processes that act to reorient the MTOC is unknown. To address this question, MTOC reorientation was imaged in living wound edge fibroblasts expressing GFP-tubulin. Surprisingly, it was observed that MTOC reorientation occurred by a major rearward movement of the nucleus, while the MTOC remained immobile. Nuclear movement was driven by actin retrograde flow and was myosin II-dependent. Cdc42 was necessary and sufficient to activate nuclear movement and myosin phosphorylation, and myotonic dystrophy kinase-related Cdc42-binding kinase (MRCK) (28) was identified as the Cdc42 effector that stimulates myosin phosphorylation and activates rearward nuclear movement. Strikingly, the previously implicated factors, dynein, Par6, and PKCξ, did not participate in nuclear movement, but instead contributed to MTOC reorientation by maintaining the MTOC at the cell centroid. Results show that MTOC reorientation is established by active movement of the nucleus, rather than the MTOC, and suggest that nuclear positioning is an initial polarizing event in migrating cells.

Introduction

The microtubule organizing center (MTOC) is reoriented between the nucleus and the leading edge in many migrating cells and contributes to directional migration. Models suggest that the MTOC is moved to its position during reorientation. By direct imaging of wound edge fibroblasts after triggering MTOC reorientation with soluble factors, we found instead that the nucleus moved away from the leading edge to reorient the MTOC, while the MTOC remained stationary. Rearward nuclear movement was coupled with actin retrograde flow and was regulated by a pathway involving Cdc42, MRCK, myosin and actin. Nuclear movement was unaffected by the inhibition of dynein, Par6 or PKCξ, yet these components were essential for MTOC reorientation as they maintained the MTOC at the cell centroid. These results show that nuclear repositioning is an initial polarizing event in migrating cells and that the positions of the nucleus and the MTOC are established by separate regulatory pathways.

Materials and Methods

Reagents

BB was from Tocris (Ellisville, Mo.). Rhodamine-phalloidin and GST-tagged Rho, Rac and Cdc42 proteins were from Cytoskeleton, Inc. (Denver, Colo.). pEGFP-tubulin was from Clontech (Palo Alto, Calif.). Unless noted, all other chemicals were from Sigma.

mPar6 (full length mouse Par-6 in pFLAG CMV2) was provided by T. Pawson (U. Toronto, Canada) (30). KdPKCξ (Kinase dead PKCξ, with a K to R mutation in the ATP binding site) was provided by I. Weinstein (Columbia U., NY) (50). Flag-MRCK-WT (wild type full length Rat MRCKa), HA-MRCK-TM (full length MRCKa with K106A, H1579A and H1582A mutations) and Flag-MRCK-CPC (truncated MRCKa, aa930-1492) were in pXJ40 and were provided by T. Leung (Glaxo-IMCB Group, Singapore) (8, 28). hPAK1(83-149) in pCMV6myc was provided by G. Bokoch (Scripps Inst., CA) (61). L61Cdc42 in pKHA was provided by R. Cerione (Cornell U., NY).

ξ-tubulin and Flag (M2) mouse mAbs were from Sigma. Pericentrin rabbit antibody was from Covance. Tyrosinated a-tubulin rat mAb (YL1/2) (25) was from European Collection of Animal Cell Cultures (Salisbury, UK). Phospho-Ser19 MLC mAb was from Y. Sasaki (Asahi Chemical Industry Co., Shizuoka, Japan) (44). β-catenin mouse and rabbit antibodies were from Zymed (San Francisco, Calif.). Myc (9E10) mAb was from Santa Cruz (Santa Cruz, Calif.). Flag rabbit antibody was from Affinity Bioreagents (Golden, Colo.). HA (12CA5) mAb was from Roche (Indianapolis, Ind.).

Cell Culture and Monolayer Wounding

NIH-3T3 cells were cultured in DMEM with 10% calf serum, serum starved for 2 days and wounded as previous described (9, 21).

Preparation of 3T3-GFPTub Cell Line

NIH-3T3 cells were transfected with EGFP-tubulin plasmid using Lipofectamine (Invitrogen, CA). After 2 days, cells were replated in culture medium with 1 mg/ml G418 and clones were selected. Individual clones expressed ˜10% of level of endogenous tubulin and exhibited normal proliferation, migration, and MTOC reorientation. For most experiments, a single clonal line was used. However, several clonal lines exhibited similar nuclear movement and MTOC reorientation.

DNA and Protein Microinjections

Plasmid DNA was purified using Plasmid Midi Kit (Qiagen, Valencia, Calif.) and was microinjected into nuclei as described (38). Expression was detectable within 1-2 hr. Proteins were microinjected as described (9, 39). DIC mAb 74.1 (from K. Pfister, U. of Virginia), was microinjected at 10 mg/ml and Cdc42, Rac and Rho GTPases were microinjected at 1 mg/ml. For double microinjections, 100 μg/ml DNA (MRCK-WT or MRCK-CPC) was injected into the nucleus with 2 mg/ml fluorescein-ovalbumin. After 30 min, labelled cells were injected with DIC mAb, L61Cdc42 or human IgG as a control.

Time-Lapse Microscopy

3T3-GFPTub cells were grown on 35 mm dishes with glass coverslip bottoms (MatTek Corp.). Confluent monolayers were starved for 24h with HBSS (Gibco) containing essential and non-essential MEM amino acids (Gibco), 2.5 g/l glucose, 2 mM glutamine, 1 mM sodium pyruvate, and 10 mM HEPES, pH 7.4. Monolayers were wounded and transferred to a Nikon TE300 microscope, equipped with a heated (34° C.) chamber and a 60X (1.4 NA) plan apo objective (Nikon). Cells were imaged for ˜30 min before adding LPA (2 μM) or serum (1%) to activate MTOC reorientation. Alternatively, multiple fields were imaged using an XY stage (Prior) and a 40X (0.6 NA) plan fluor objective (Nikon). Fluorescence and phase contrast images were collected with TEA/CCD (Princeton Instruments) or Coolsnap HQ (Roper Scientific) cameras controlled by Metamorph software (Universal Imaging Corporation).

Immunofluorescence

Coverslips were fixed in either −20° C. methanol or paraformaldehyde as previously described (38, 39). Secondary antibodies were from Jackson ImmunoResearch Laboratories.

Determination of Nucleus and MTOC Position

Phase contrast and fluorescence images of cells stained for pericentrin or γ-tubulin, β-catenin, DNA, expression tag, MTs. (Tyr tubulin) or injection marker were acquired as described (38, 39). MTOC reorientation was determined as previously described (39). For analysis of nucleus and MTOC position, images were pseudo-colored, aligned with the wound parallel to the X axis and then combined using Metamorph software. The cell perimeter was drawn over the cell-cell contacts (β-catenin, expression tags or injection markers) and the wound edge (Tyr-tubulin or injection markers) and the cell centroid was calculated using Metamorph software. Similarly, the nuclear perimeter was drawn and the centroid of the nucleus calculated. A vector representing the distances from the centroid of the nucleus and the MTOC (γ-tubulin or pericentrin staining and MT focus) to the cell centroid was drawn and resolved into X and Y coordinates (parallel and perpendicular to the leading edge, respectively). To allow comparison between cells, measurements were normalized to cell size. Only the Y coordinate was used in plots as nuclear or MTOC position along the X axis did not change. At least 30 cells from 3 independent experiments were analysed for each condition and error bars in plots are SEM.

Phospho-Ser19 MLC Quantification

Total cell extracts prepared in SDS sample buffer were analyzed by western blotting as described (38). For quantification of pSer19-MLC immunofluorescence, average cellular fluorescence intensity was determined with Metamorph software after subtracting background fluorescence (determined for an adjacent cell-free area). Data is from at least two independent experiments and error bars in plots are SEM.

Speckle Microscopy

Serum-starved 3T3 cells were wounded and cells at the wound edge were microinjected with 0.25 mg/ml X-Rhodamine actin (generously provided by Clare Waterman-Storer, Scripps Inst., CA) as described (45). Images of actin speckles in cells before and after LPA treatment were acquired as for recordings of MTs.

Results

### Rearward Nuclear Rather than Forward MTOC Movement Reorients the MTOC

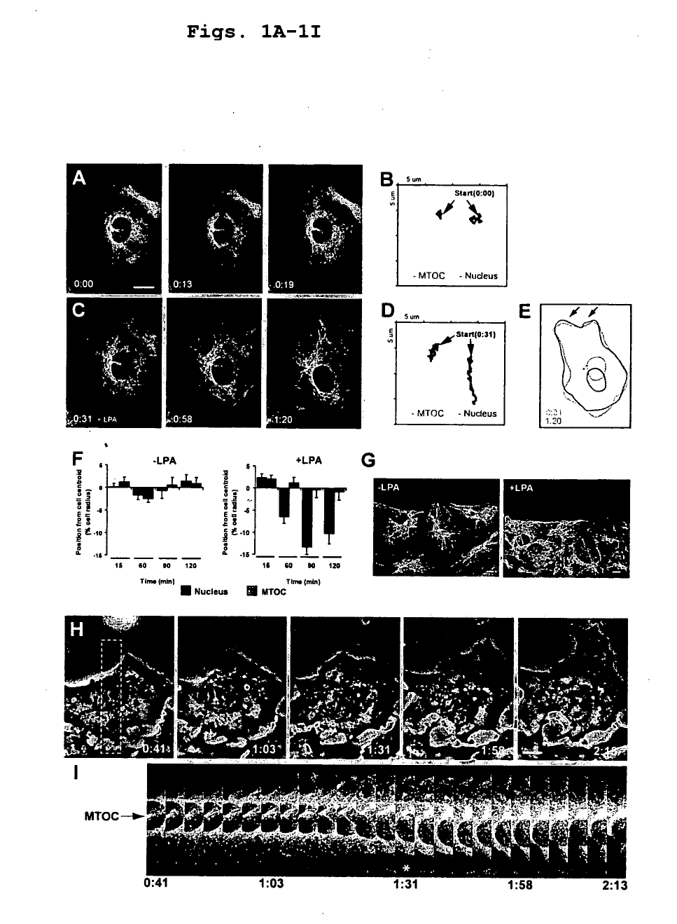

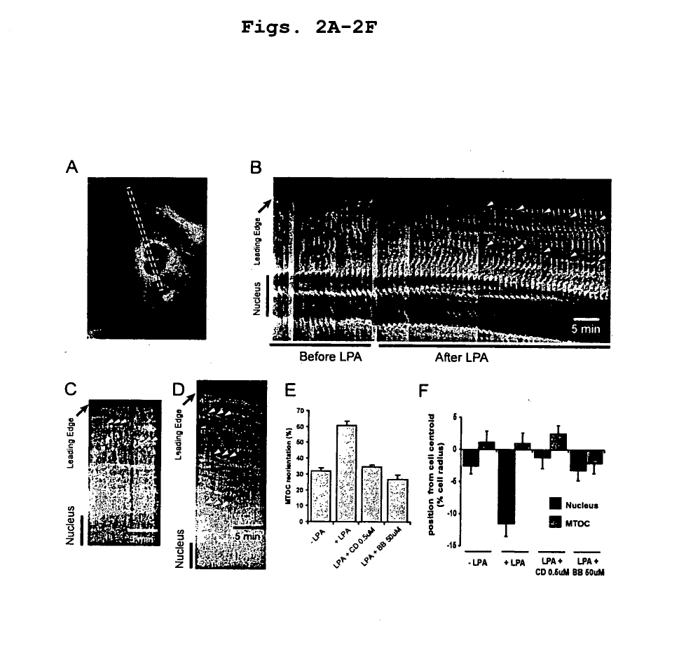

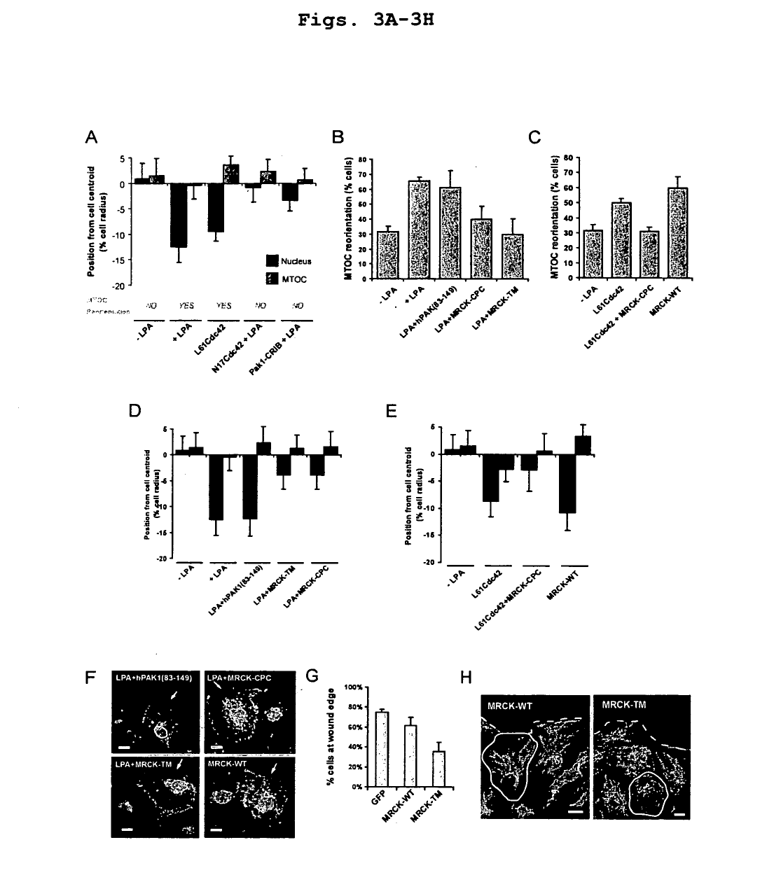

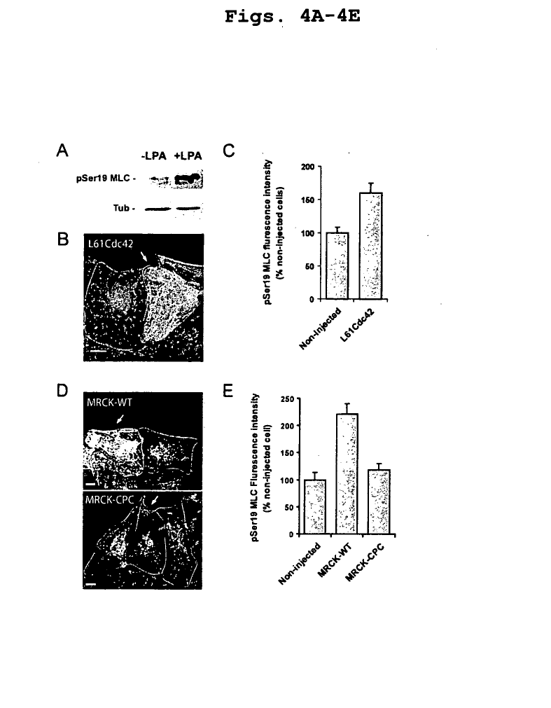

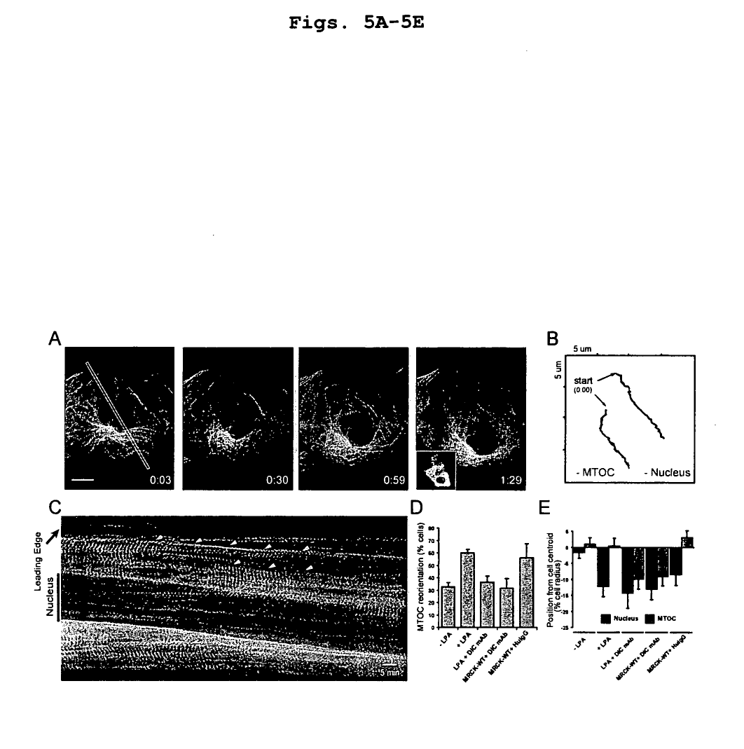

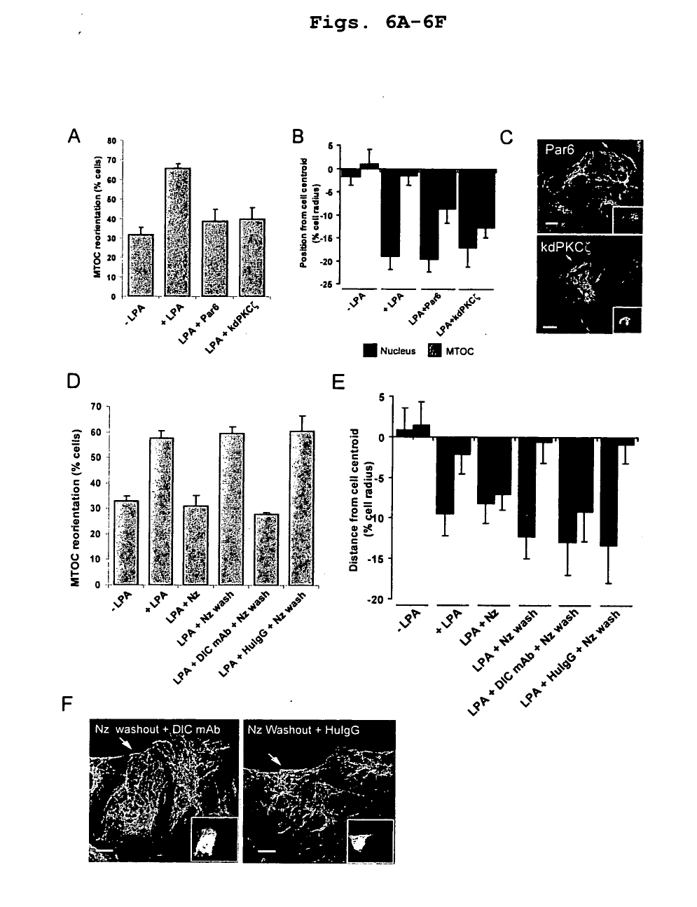

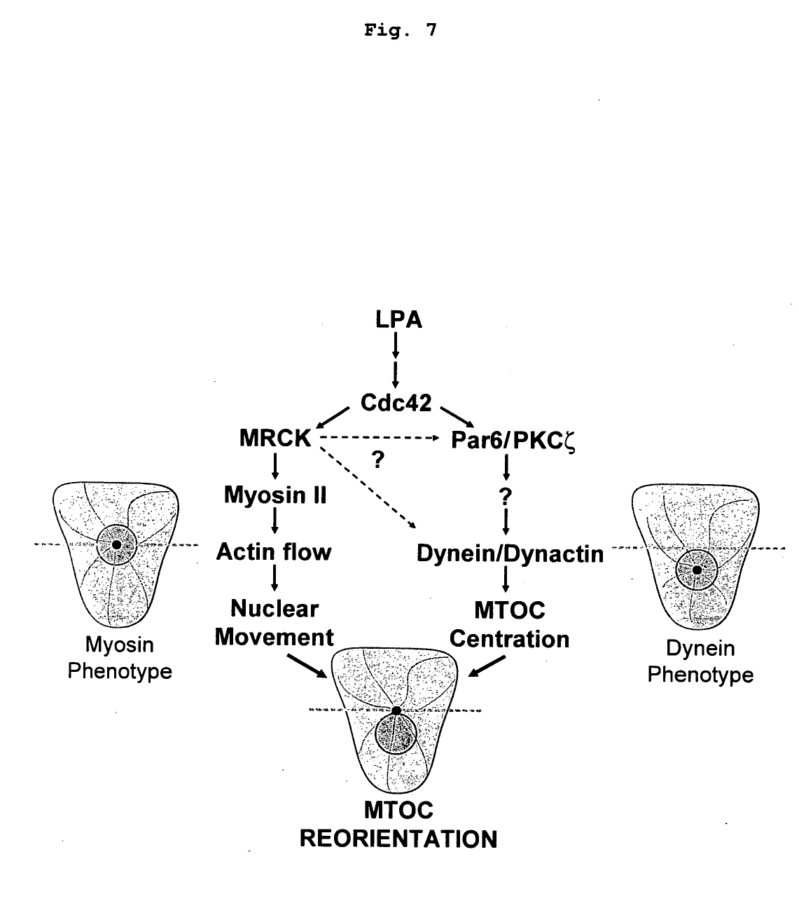

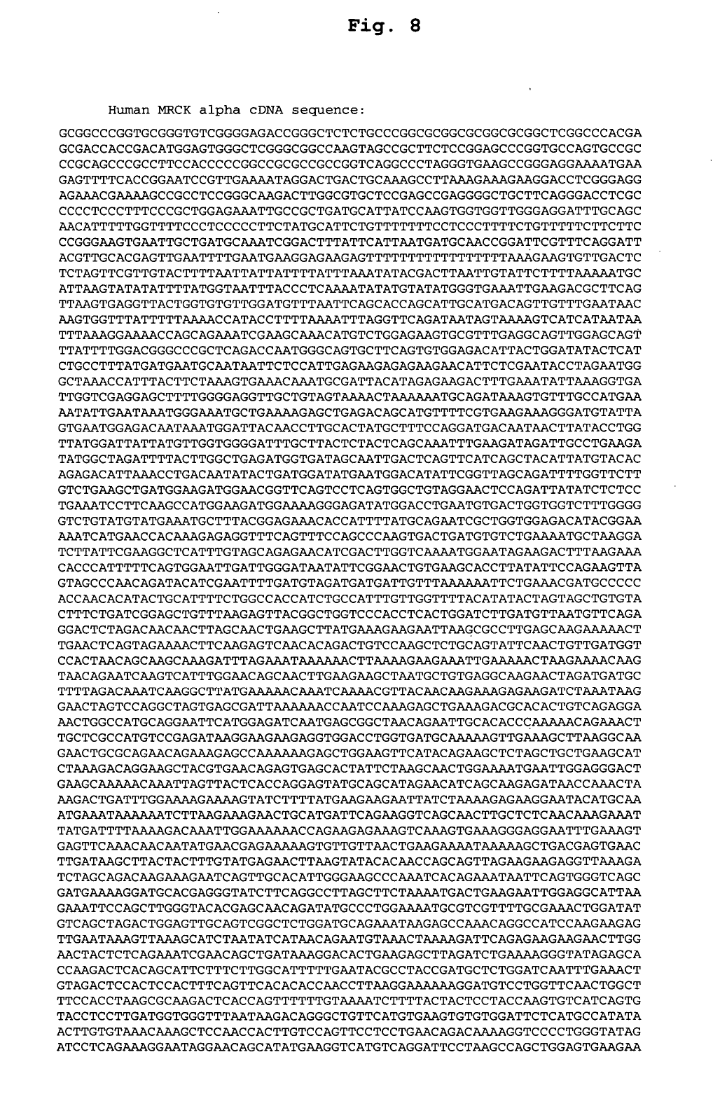

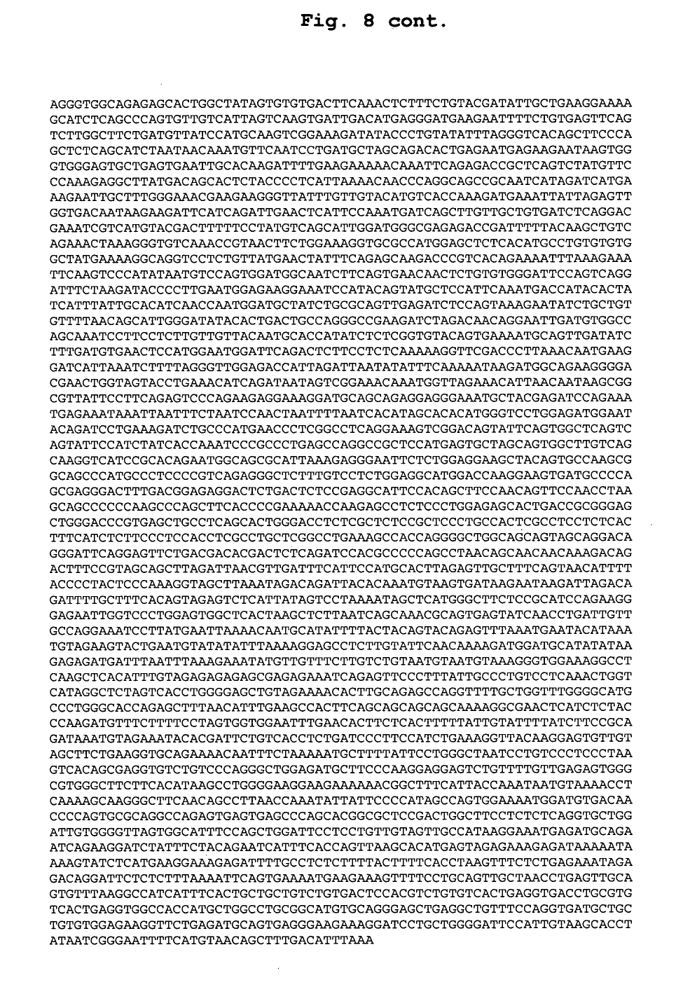

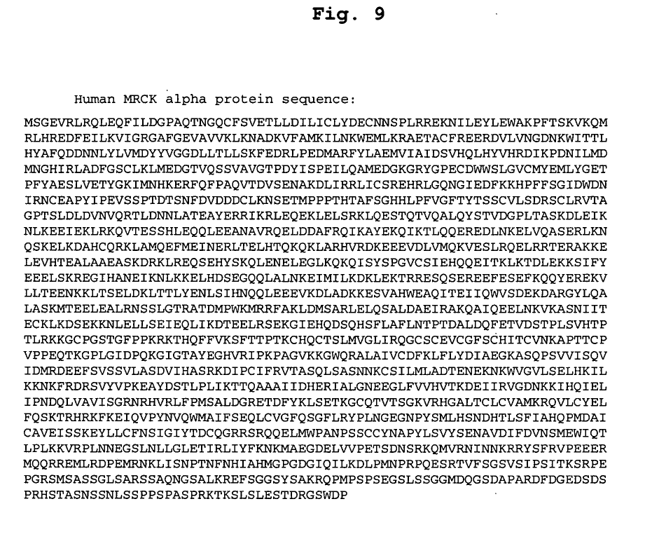

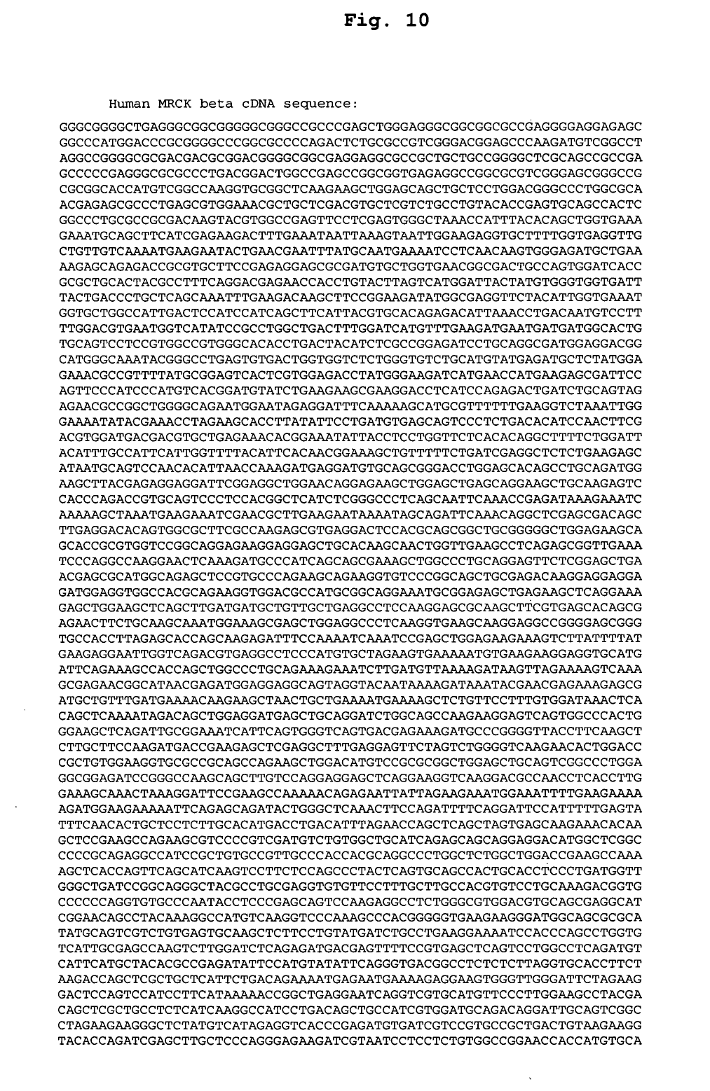

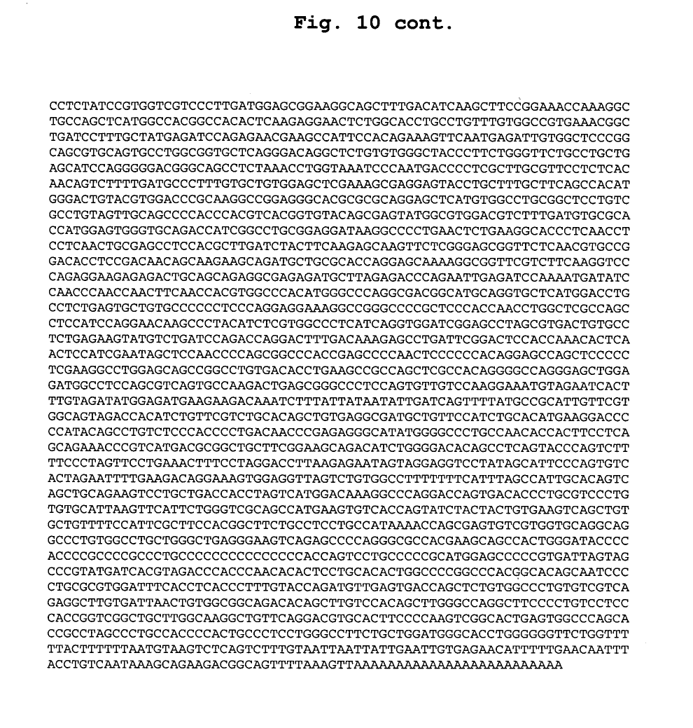

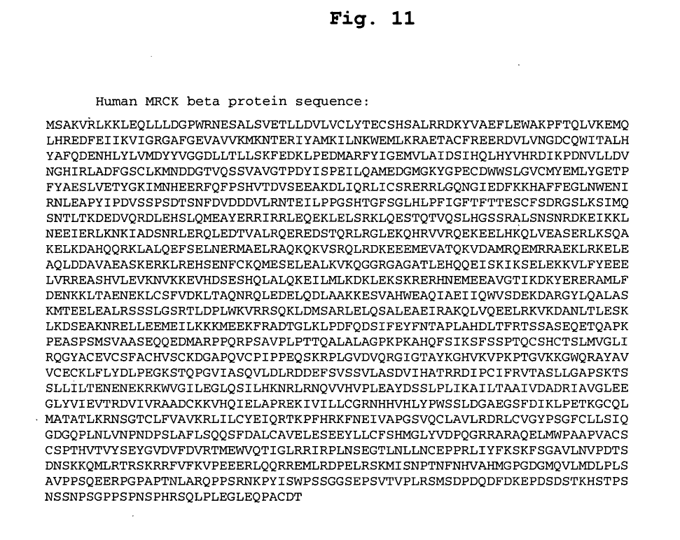

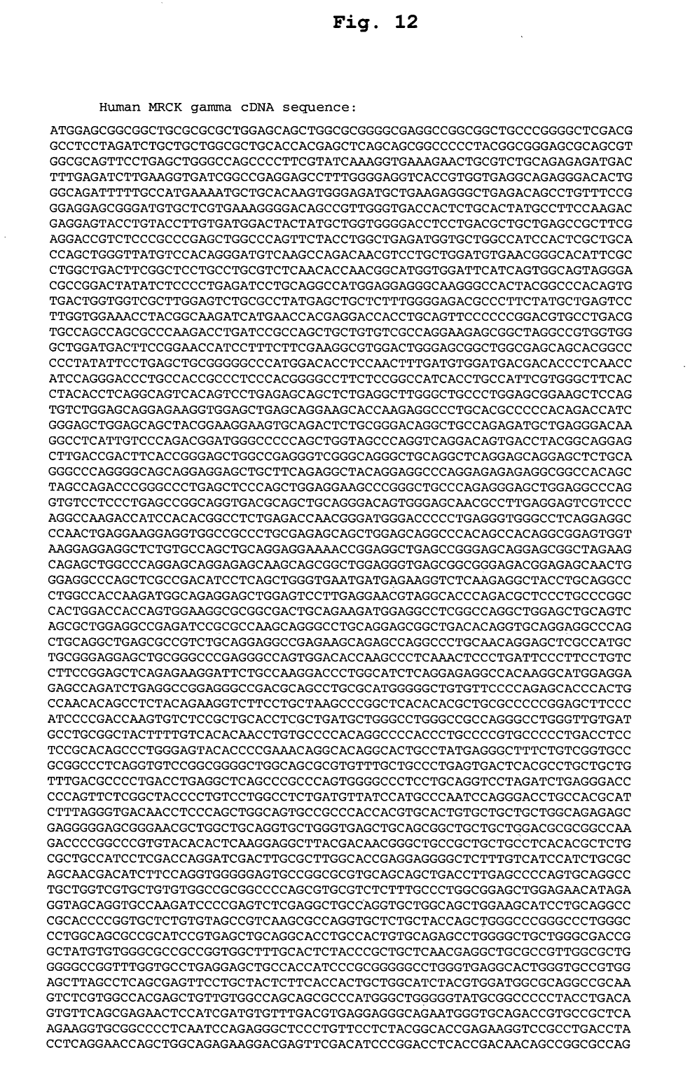

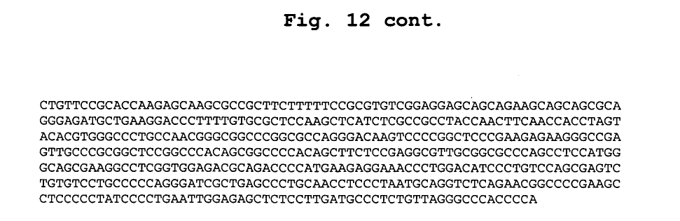

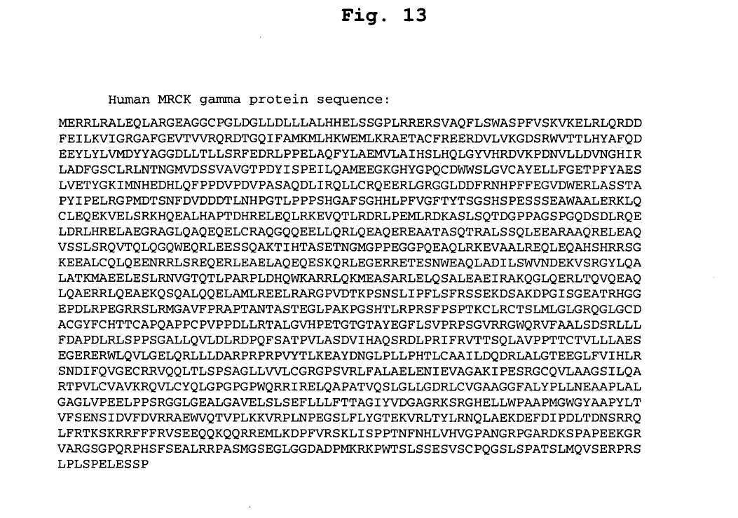

NIH 3T3 cell lines stably expressing GFP-α-tubulin (3T3-GFPTub) were prepared to visualize the MTOC in living cells. In these cells, the MTOC is clearly detected as a single (or occasionally double) spot of fluorescence near the cell center from which MTs emanate (FIG. 1; Suppl. Movie 1). As previously observed for parental NIH-3T3 cells, wounding alone did not induce MTOC reorientation in serum-starved 3T3-GFPTub monolayers (39). This allowed MTOC position before and after stimulating MTOC reorientation by adding serum or the specific serum factor LPA to be monitored (39). After wounding, but before the addition of LPA, neither the MTOC nor the nucleus moved from their starting positions near the cell center (FIGS. 1A, B; Suppl. Movie S1). After addition of LPA, the nucleus moved rearward (away from the wound edge), while the MTOC remained stationary or moved slightly toward the rear (FIGS. 1C, D; Suppl. Movie S2). Overlaying outlines of the cell, the nucleus and the MTOC showed that the rearward nuclear movement occurred relative to the leading edge (FIG. 1E). Rearward movement of the nucleus was the predominant movement that resulted in MTOC reorientation in every live recording examined (N=9). MTOC reorientation occurred 80±28 min after the addition of LPA with the nucleus moving at a velocity of 0.28±0.09 μm/min (N=8).

The involvement of nuclear movement in MTOC reorientation in larger numbers of cells was monitored by analyzing the position of the nucleus and the MTOC relative to the cell centroid in fixed NIH 3T3 cells. This analysis showed that the MTOC and the nucleus remained in the centroid of untreated, wound-edge cells for up to 2 hr after wounding (FIG. 1F). In contrast, with LPA treatment, the nucleus was located at increasingly rearward positions from the cell centroid, reaching a maximum average position of 13.3±1.7% of the cell radius from the cell centroid at 90 min. During this interval the MTOC remained near the cell centroid (FIG. 1F). The change in position of the nucleus, but not the MTOC, can be appreciated in representative images of the cells +/−LPA treatment (FIG. 1G). Thus, analysis of both living and fixed cells shows that MTOC reorientation results from rearward movement of the nucleus rather than forward movement of the MTOC. Similar results were seen in NRK fibroblasts (data not shown).

LPA treatment of starved NIH 3T3 cells induces MTOC reorientation and MT stabilization, but it does not induce cell protrusion and migration (FIG. 1E and (9, 39)). To determine if rearward nuclear movement reoriented the MTOC during active cell migration, starved 3T3-GFPTub cells were wounded and treated with calf serum, which induces a complete migration response in starved 3T3 cells (21). Serum triggered cell protrusion and migration as expected, and as was observed with LPA, the nucleus moved away from the leading edge, while the MTOC remained stationary, resulting in reorientation of the MTOC (FIGS. 1H, I; Suppl. Movie S3). In 93% of the serum-stimulated cells in which the nucleus and the MTOC(N=28) were directly imaged, rearward nuclear movement was responsible for reorienting the MTOC. The rate of nuclear movement triggered by serum was similar to that triggered by LPA (0.26±0.12 μm/min; N=15). In most cells the MTOC reoriented before sustained cell translocation (FIG. 1H; compare frame 1:03 with 1:31). As cells continued migration into the wound at later times (>2 hr), the nucleus moved forward with the MTOC and the MTOC remained near the cell centroid, consistent with earlier studies (15). Thus, rearward nuclear movement also reorients the MTOC in migrating cells.

### Retrograde Flow of Actin is Involved in Nuclear Movement

The velocity of rearward nuclear movement was similar to that of actin and MT retrograde flow (33, 45, 58).

Kymographs were analyzed to see whether retrograde flow was activated by LPA and coupled with nuclear movement. Kymographs showed that LPA triggered rearward movement of some MTs in the lamella (FIGS. 2A, B). The slopes of the lines in the kymographs representing rearward moving MTs and nuclei were nearly identical, indicating that both were moving at the same velocity (MTs: 0.23 μm/min; nucleus: 0.26 μm/min). As MT retrograde flow is driven by actin retrograde flow (45, 58), this result suggests that actin retrograde flow might be responsible for the rearward movement of the nucleus. It also suggests that LPA either triggers actin retrograde flow itself or alternatively, coupling of the nucleus (and MTs) to constitutively active actin retrograde flow.

To test these possibilities, fluorescent “speckle” analysis of F-actin in starved wound edge cells microinjected with low concentrations of X-rhodamine actin (R-actin) was used (45). Before LPA treatment, actin speckles in the lamella did not exhibit retrograde flow for periods up to 15 min (FIG. 2C). After LPA treatment, actin speckles moved rearward at a rate of 0.25±0.04 μm/min (N=6) and this movement coincided with rearward movement of the nucleus (FIG. 2D).

Whether actin retrograde flow was necessary for rearward movement of the nucleus was tested next. Inhibition of myosin II with blebbistatin (BB) or interference of actin with cytochalasin D (CD) is known to block actin retrograde flow (40, 58). Pre-treatment of starved wound edge cells with 0.5 μM CD or 50 μM BB, blocked both MTOC reorientation and rearward nuclear movement induced by LPA, without affecting the position of the MTOC (FIGS. 2E, F). CD and BB blocked LPA-induced changes in the actin cytoskeleton as expected (FIG. 2G). Lower concentrations of CD had no effect on MTOC reorientation as previously observed (39).

### Cdc42 is Necessary and Sufficient for Rearward Nuclear Movement

Cdc42 is activated upon LPA addition to starved NIH-3T3 cells and is critical for MTOC reorientation in these cells (39). Whether Cdc42's function in MTOC reorientation included regulation of nuclear movement was tested. Microinjected constitutively active L61Cdc42 protein induced rearward nuclear movement in starved wound-edge NIH-3T3 cells and this resulted in MTOC reorientation (FIG. 3A). Conversely, LPA-induced rearward nuclear movement was blocked by microinjected dominant-negative N17Cdc42 protein or by the CRIB domain of PAK1 (PAK-CRIB), which inhibits both Cdc42 and Rac (46) (FIG. 3A). With all these treatments, the MTOC remained near the cell centroid. These results show that Cdc42 is both necessary and sufficient for the rearward nuclear movement that generates MTOC reorientation.

### MRCK is Involved in MTOC Reorientation, Rearward Nuclear Movement and Cell Migration

Next, the Cdc42 effector(s) involved in regulating nuclear movement were identified. As rearward nuclear movement involved myosin-dependent actin retrograde flow, focus was placed on two Cdc42 effectors that might regulate this activity. Pak1 is a Cdc42 regulated kinase and while it tends to decrease myosin II activation (4), there is evidence that it regulates actin retrograde flow in epithelial cells (59). MRCK is a Cdc42 effector that is capable of activating myosin II by phosphorylating Ser19 of the myosin light chain (MLC) (28).

Starved wound edge cells microinjected with the N-terminal autoregulatory domain of Pak1 (hpak1(83-149)), which inhibits Pak1 activity in vivo (59), still reoriented their MTOC and moved their nuclei rearward upon LPA stimulation (FIGS. 3B, D, F). These results suggest that Pak1 is not involved in either MTOC reorientation or rearward nuclear movement.

In contrast, MRCK was important for both MTOC reorientation and rearward nuclear movement. Two different dominant negative MRCK constructs were used: MRCK-CPC, which lacks both the kinase domain and GTPase binding domain (GBD), and MRCK-TM, a full length construct with inactivating mutations in the kinase domain and the GBD to disrupt Cdc42 binding (8, 28). Expression of either dominant negative MRCK blocked MTOC reorientation induced by LPA by blocking the rearward movement of the nucleus (FIGS. 3B, D, F). MRCK-TM did not have a major effect on LPA-induced changes in the actin cytoskeleton (FIG. 3G). MRCK-CPC also inhibited MTOC reorientation and nuclear movement induced by L61Cdc42 (FIGS. 3C, E), indicating that MRCK functioned downstream of Cdc42. Expression of wild type MRCK in starved wound edge cells, without any other treatment, induced MTOC reorientation and nuclear movement while maintaining the MTOC at the cell centroid (FIGS. 3C, E, F). These results show that MRCK is both necessary and sufficient for MTOC reorientation and nuclear movement.

MRCK has not previously been implicated in cell migration. Because these results show that MRCK regulates MTOC reorientation and nuclear position in migrating cells, this possibility was tested. Dominant negative MRCK-TM or wild type MRCK were expressed in starved wound edge cells by DNA microinjection and then the cells were stimulated to migrate with serum. Control GFP or wild type MRCK expressing cells kept up with the wound edge (FIGS. 3H, I) indicating that these proteins had no effect on cell migration. In contrast, MRCK-TM expressing cells tended to fall behind the wound edge (FIGS. 3H, I) indicating that their migration was inhibited. These results indicate that MRCK participates in cell migration and are consistent with MRCK's role in regulating MTOC reorientation and nuclear position.

### Myosin II is Activated by Cdc42 and MRCK during MTOC Reorientation and Rearward Nuclear Movement

The results with MRCK and BB lead to the prediction that myosin II is activated during MTOC reorientation and rearward nuclear movement. LPA triggers myosin II activation by stimulating phosphorylation of Ser 19 of MLC (23) and this result was confirmed in our starved NIH 3T3 cells treated with LPA (FIG. 4A). LPA is known to activate Rho and Rho kinase and these activate myosin II, however, Rho and Rho kinase are not involved in MTOC reorientation (39). To test whether Cdc42 and MRCK might also regulate myosin II, pSer19 MLC antibody was used to immunofluorescently stain cells that had been injected with active L61Cdc42 or MRCK. Starved wound edge cells injected with L61Cdc42 contained substantially increased pSer19 MLC compared to noninjected cells (FIGS. 4B, C). Expression of wild type MRCK in starved wound edge cells also increased pSer19 MLC while dominant negative MRCK-CPC had no effect (FIGS. 4D, E). These results show that Cdc42 and MRCK are sufficient to activate myosin II during MTOC reorientation and rearward nuclear movement.

### Dynein Contributes to MTOC Reorientation by Maintaining the MTOC at the Cell Centroid

Previously, it was shown that inhibition of cytoplasmic dynein in 3T3 fibroblasts blocked LPA and L61Cdc42 triggered MTOC reorientation (39). Cytoplasmic dynein localizes to the ends of MTs projecting toward the leading edge in 3T3 fibroblasts (11), yet the role of dynein in MTOC reorientation is unclear. To test whether dynein was involved in nuclear movement, movies of starved wound edge 3T3-GFPTub cells injected with an inhibitory dynein intermediate chain monoclonal antibody (DIC mAb) (39) and then stimulated MTOC reorientation with LPA were prepared. Surprisingly, rearward nuclear movement still occurred in cells injected with DIC mAb. However, the MTOC no longer remained at the cell centroid and instead moved together with the nucleus away from the leading edge (FIGS. 5A, B; Suppl. Movie S4). The rearward displacement of the MTOC with the nucleus prevented MTOC reorientation. Kymographic analysis showed that retrograde flow of MTs in the lamella was still coupled with nuclear movement (FIG. 5C). Analysis of the positions of the nucleus and the MTOC in fixed populations of LPA-treated cells that had been injected with DIC mAb confirmed that dynein inhibition displaced the MTOC rearward of the cell centroid without interfering with rearward movement of the nucleus (FIG. 5E). Injection of DIC mAb, but not control HuIgG, also blocked MTOC reorientation induced by wild type MRCK and as with LPA, this was due to failure to maintain the MTOC at the cell centroid (FIGS. 5D, E). These results show that dynein is not involved in the rearward movement of the nucleus, but instead plays a role in maintaining the MTOC at the cell centroid.

### Par6 and Atypical Protein Kinase Cξ Function with Dynein to Maintain the MTOC at the Cell Centroid

Previous work has implicated the Par6-atypical PKCξ complex as a Cdc42 effector involved in MTOC reorientation in astrocytes and endothelial cells (13, 56). However, these studies did not determine whether Par6 and PKCξ regulated nuclear or MTOC position during MTOC reorientation. Expression of full length Par6 or a kinase-dead mutant of PKCξ (kdPKCξ), both of which were previously shown to inhibit MTOC reorientation (13), blocked LPA-stimulated MTOC reorientation in starved wound-edge fibroblasts (FIG. 6A). Analysis of the position of the nucleus and the MTOC showed that the movement of the nucleus rearward of the cell centroid was unaffected by expression of Par-6 or kdPKCξ (FIGS. 6B, C). However, expression of Par-6 or kdPKCξ led to a displacement of the MTOC from its position at the centroid (FIGS. 6B, C), the same phenotype as observed with dynein inhibition. These results show that Par-6 and PKCξ do not regulate rearward nuclear movement, but instead are likely to function together with dynein to maintain the MTOC at the cell centroid.

### Dynamic MTs Coupled with Dynein Maintain the MTOC at the Cell Centroid

Models for dynein's role in positioning nuclei and spindles posit that dynein is important to tether and pull MTs at the cell cortex. To determine whether dynein might tether MTs to maintain the MTOC at the cell centroid during MTOC reorientation, it was first asked whether dynamic MTs were important for MTOC reorientation. Starved 3T3 cell monolayers were wounded and treated with LPA for 2h in the presence of 100 nM nocodazole, which inhibits dynamic MTs in fibroblasts without affecting overall MT distribution (FIG. 6F) (32). This treatment blocked MTOC reorientation by interfering with MTOC centration but not with nuclear movement (FIGS. 6D, E). Thus, dampening MT dynamics has a “dynein phenotype” on MTOC reorientation. Upon nocodazole wash out, the MTOC returned to the cell center. These results show that dynamic MTs are necessary for maintaining the MTOC at the cell centroid during MTOC reorientation.

The requirement for dynamic MTs in MTOC centration could reflect a need for MTs to be tethered to cortical factors such as dynein. To test this idea, whether dynein was necessary for the re-centration of the MTOC observed after nocodazole washout was explored. Microinjection of DIC mAb into nocodazole- and LPA-treated cells blocked MTOC recentration when nocodazole was subsequently washed out (FIG. 6D-F). The rearward position of the nucleus was not affected by DIC mAb. Control HuIgG had no effect (FIG. 6D-F). Thus, MT dynamics alone are not sufficient to re-center the MTOC after nocodazole washout; dynein is also required. These results suggest that the most likely role for dynein in centering the MTOC is in tethering MTs.

Discussion

It is shown that MTOC reorientation results from nuclear movement away from the leading edge while the MTOC is maintained at the cell centroid. These results do not support previous models that propose that the MTOC moves to a position between the nucleus and the leading edge during MTOC reorientation (14, 22, 47). Instead, this data supports a new model for MTOC reorientation in which Cdc42 regulates two major activities through distinct effectors: MRCK-regulated actinmyosin retrograde flow to move the nucleus rearward and Par6-PKCξ-regulated dynein centration of the MTOC to prevent the MTOC from being swept rearward with the nucleus (FIG. 7). Interfering with only one of the two pathways prevents MTOC reorientation, but with different inhibitory phenotypes. Thus, if nuclear movement is blocked, both MTOC and nucleus are positioned at the cell centroid, whereas if MTOC centration is blocked, both MTOC and nucleus are rearward of the cell centroid (FIG. 7).

It is generally thought that the rearward position of nuclei commonly observed in migrating cells develops as a consequence of cell extension. However, these results clearly show that cells possess an active mechanism for repositioning the nucleus and that this functions independently of cell extension and for the most part, of the factors regulating the position of the MTOC. As observed in cells stimulated with LPA or with serum, the rearward positioning of the nucleus can be the initiating event to establish the relative orientation of the MTOC and nucleus. Although the importance of MTOC reorientation may lie in repositioning the MT array and associated Golgi apparatus, perhaps to direct membrane precursors to the leading edge, this data suggest another possibility. Rearward nuclear positioning may ensure that the nucleus is in the proper orientation to be pulled forward as the cell extends. Indeed, in migrating neuronal cells, MTOC movement toward the leading process precedes that of the nucleus (51). In migrating fibroblasts the events may be coordinated so that the MTOC and nucleus move forward together, yet like neurons, the nucleus lags the MTOC. Additional studies are needed to test this idea, but the dynein dependence of forward movement of the MTOC after nocodazole washout (this study) combined with the localization of dynein at the leading edge (11) point to pulling forces on the MTOC and nucleus being exerted from the front.

This data points to a new actin-based mechanism for nuclear positioning. The most likely mechanism would involve Cdc42-MRCK regulation of actin-myosin retrograde flow. Such a mechanism is clearly distinct from dynein-dependent nuclear positioning mechanisms. There is some precedent for actin-dependent positioning of the nucleus. In Arabidopsis, an intact actin cytoskeleton is necessary to maintain the nucleus at a fixed distance from the apex of growing root hairs and to release them upon growth arrest (24). In the syncitial Drosophila embryo, nuclei disperse along the anterior-posterior axis in an actinmyosin dependent fashion although it is unclear whether actin-myosin directly move the nuclei (43, 57). Testing whether Cdc42 or MRCK regulate nuclear position in these systems will be interesting.

The control of nuclear position by MRCK through its regulation of actin-myosin represents a new function for MRCK. MRCK was first identified as a putative Cdc42 effector in Drosophila (where it is called Gek, for Genghis Khan) (31) and later shown to be a Cdc42 effector in mammalian cells (28). Drosophila Gek mutants are lethal and Gek is essential for proper oogenesis, probably due to its regulation of actin organization. MRCK phosphorylates MLC and the myosin binding subunit of myosin phosphatase in vitro (28, 54), and increased myosin phosphorylation was observed in cells expressing MRCK (FIG. 4). MRCK also phosphorylates moesin (35) and this may also contribute to nuclear migration. As overexpressed MRCK is sufficient for full MTOC reorientation, it will be interesting to see whether MRCK is also involved in the regulation of the MTOC centration pathway (FIG. 7).

An interesting question is how actin retrograde flow drives nuclear movement. Two general models can be envisioned: bulk actin retrograde movement simply pushes the nucleus rearward (bulldozer model) or the nucleus is specifically linked to actin filaments and when these move, the nucleus moves with them (conveyor belt model). Accumulation of actin filaments on the trailing side of moving nuclei was not detected, which does not support the first model. Consistent with the second model is the tight correlation between the initiation of actin retrograde flow and nuclear movement. If the conveyor belt model is correct, specific linking proteins may attach the nucleus to actin. Candidate linkers are the conserved Syne/ANC-1 proteins that have actin-binding calponin homology domains and contribute to nuclei positioning in a number of systems (52).

It has been shown that dynein-dynactin and Par6-PKCξ are necessary for MTOC reorientation (12, 39, 56). These new findings show that these proteins are only involved in the MTOC cell centroid maintenance pathway and not in the nuclear movement pathway. The maintenance of the MTOC at the cell centroid in both migrating and non-migrating cells has been proposed to result from pulling and/or pushing forces exerted on the MT array by cortical dynein and/or MT dynamics (5, 11). The dyneindependent re-centering of the MTOC after nocodazole wash out is consistent with pulling forces maintaining the MTOC at the cell center. Dynein and dynactin are enriched at the leading edge in migrating fibroblasts (11) where they may oppose forces exerted on the MTOC by actin retrograde flow and/or the nucleus. Dynein at cell-cell contacts may also contribute to MTOC centration (29).

### REFERENCES

1. Adames, N. R., and Cooper, J. A. (2000). Microtubule interactions with the cell cortex causing nuclear movements in Saccharomyces cerevisiae. J Cell Biol 149, 863-874.
2. Ahringer, J. (2003). Control of cell polarity and mitotic spindle positioning in animal cells. Curr Opin Cell Biol 15, 73-81.
3. Bergmann, J. E., Kupfer, A., and Singer, S. J. (1983). Membrane insertion at the leading edge of motile fibroblasts. Proc Natl Acad Sci USA 80, 1367-1371.
4. Bokoch, G. M. (2003). Biology of the p21-activated kinases. Annu Rev Biochem 72, 743-781.
5. Burakov, A., Nadezhdina, E., Slepchenko, B., and Rodionov, V. (2003). Centrosome positioning in interphase cells. J Cell Biol 162, 963-969.
6. Busson, S., Dujardin, D., Moreau, A., Dompierre, J., and De Mey, J. R. (1998). Dynein and dynactin are localized to astral microtubules and at cortical sites in mitotic epithelial cells. Curr Biol 8, 541-544.
7. Carminati, J. L., and Stearns, T. (1997). Microtubules orient the mitotic spindle in yeast through dynein-dependent interactions with the cell cortex. J Cell Biol 138, 629-641.
8. Chen, X. Q., Tan, I., Leung, T., and Lim, L. (1999). The myotonic dystrophy kinase-related Cdc42-binding kinase is involved in the regulation of neurite outgrowth in PC12 cells. J Biol Chem 274, 19901-19905.
9. Cook, T. A., Nagasaki, T., and Gundersen, G. G. (1998). Rho guanosine triphosphatase mediates the selective stabilization of microtubules induced by lysophosphatidic acid. J Cell Biol 141, 175-185.
10. Dujardin, D. L., and Vallee, R. B. (2002). Dynein at the cortex. Curr Opino Cell Biol 14, 44-49.
11. Dujardin, D. L., Barnhart, L. E., Stehman, S. A., Gomes, E. R., Gundersen, G. G., and Vallee, R. B. (2003). A role for cytoplasmic dynein and LIS1 in directed cell movement. J Cell Biol 163, 1205-1211.
12. Etienne-Manneville, S., and Hall, A. (2001a). Integrin-mediated activation of Cdc42 controls cell polarity in migrating astrocytes through PKCzeta. Cell 106, 489-498.
13. Etienne-Manneville, S., and Hall, A. (2001b). Integrin-mediated activation of Cdc42 controls cell polarity in migrating astrocytes through PKCξ. Cell 106, 489-498.
14. Etienne-Manneville, S. (2004). Cdc42—the centre of polarity. J Cell Sci 117, 1291-1300.
15. Euteneuer, U., and Schliwa, M. (1992). Mechanism of centrosome positioning during the wound response in BSC-1 cells. J Cell Biol 116, 1157-1166.
16. Faulkner, N. E., Dujardin, D. L., Tai, C. Y., Vaughan, K. T., O'Connell, C. B., Wang, Y., and Vallee, R. B. (2000). A role for the lissencephaly gene LIS1 in mitosis and cytoplasmic dynein function. Nat Cell Biol 2, 784-791.
17. Gonczy, P., Pichler, S., Kirkham, M., and Hyman, A. A. (1999). Cytoplasmic Dynein Is Required for Distinct Aspects of MTOC Positioning, Including Centrosome Separation, in the One Cell Stage Caenorhabditis elegans Embryo. J Cell Biol 147, 135-150.
18. Gotlieb, A. I., May, L. M., Subrahmanyan, L., and Kalnins, V. I. (1981). Distribution of microtubule organizing centers in migrating sheets of endothelial cells. J Cell Biol 91, 589-594.
19. Gregory, W. A., Edmondson, J. C., Hatten, M. E., and Mason, C. A. (1988). Cytology and neuron-glial apposition of migrating cerebellar granule cells in vitro. J Neurosci 8, 1728-1738.
20. Gundersen, G. G., and Bulinski, J. C. (1988). Selective stabilization of microtubules oriented toward the direction of cell migration. Proc Natl Acad Sci USA 85, 5946-5950.
21. Gundersen, G. G., Kim, I., and Chapin, C. J. (1994). Induction of stable microtubules in 3T3 fibroblasts by TGF-beta and serum. J Cell Sci 107, 645-659.
22. Gundersen, G. G. (2002). Evolutionary conservation of microtubule-capture mechanisms. Nat Rev Mol Cell Biol 3, 296-304.
23. Kaibuchi, K., Kuroda, S., and Amano, M. (1999). Regulation of the cytoskeleton and cell adhesion by the Rho family GTPases in mammalian cells. Annu Rev Biochem 68, 459-486.
24. Ketelaar, T., Faivre-Moskalenko, C., Esseling, J. J., de Ruijter, N.C., Grierson, C. S., Dogterom, M., and Emons, A. M. (2002). Positioning of nuclei in Arabidopsis root hairs: an actin-regulated process of tip growth. Plant Cell 14, 2941-2955.
25. Kilmartin, J. V., Wright, B., and Milstein, C. (1982). Rat monoclonal antitubulin antibodies derived by using a new nonsecreting rat cell line. J Cell Biol 93, 576-582.
26. Kupfer, A., Louvard, D., and Singer, S. J. (1982). Polarization of the Golgi apparatus and the microtubule-organizing center in cultured fibroblasts at the edge of an experimental wound. Proc Natl Acad Sci USA 79, 2603-2607.
27. Kupfer, A., Dennert, G., and Singer, S. J. (1983). Polarization of the Golgi apparatus and the microtubule-organizing center within cloned natural killer cells bound to their targets. Proc Natl Acad Sci USA 80, 7224-7228.
28. Leung, T., Chen, X. Q., Tan, I., Manser, E., and Lim, L. (1998). Myotonic dystrophy kinaserelated Cdc42-binding kinase acts as a Cdc42 effector in promoting cytoskeletal reorganization. Mol Cell Biol 18, 130-140.
29. Ligon, L. A., Karki, S., Tokito, M., and Holzbaur, E. L. (2001). Dynein binds to beta-catenin and may tether microtubules at adherens junctions. Nat Cell Biol 3, 913-917.
30. Lin, D., Edwards, A. S., Fawcett, J. P., Mbamalu, G., Scott, J. D., and Pawson, T. (2000). A mammalian PAR-3-PAR-6 complex implicated in Cdc42/Rac1 and aPKC signalling and cell polarity. Nat Cell Biol 2, 540-547.
31. Luo, L., Lee, T., Tsai, L., Tang, G., Jan, L. Y., and Jan, Y. N. (1997). Genghis Khan (Gek) as a putative effector for Drosophila Cdc42 and regulator of actin polymerization. Proc Natl Acad Sci USA 94, 12963-12968.
32. Mikhailov, A., and Gundersen, G. G. (1998). Relationship between microtubule dynamics and lamellipodium formation revealed by direct imaging of microtubules in cells treated with nocodazole or taxol. Cell Motil Cytoskeleton 41, 325-340.
33. Mikhailov, A. V., and Gundersen, G. G. (1995). Centripetal transport of microtubules in motile cells. Cell Motil Cytoskeleton 32, 173-186.
34. Morris, N. R. (2003). Nuclear positioning: the means is at the ends. Curr Opin Cell Biol 15, 54-59.
35. Nakamura, N., Oshiro, N., Fukata, Y., Amano, M., Fukata, M., Kuroda, S., Matsuura, Y., Leung, T., Lim, L., and Kaibuchi, K. (2000). Phosphorylation of ERM proteins at filopodia induced by Cdc42. Genes Cells 5, 571-581.
36. Nobes, C. D., and Hall, A. (1999). Rho GTPases Control Polarity, Protrusion, and Adhesion during Cell Movement. J Cell Biol 144, 1235-1244.
37. O'Connell, C. B., and Wang, Y. L. (2000). Mammalian spindle orientation and position respond to changes in cell shape in a dynein-dependent fashion. Mol Biol Cell 11, 1765-1774.
38. Palazzo, A. F., Cook, T. A., Alberts, A. S., and Gundersen, G. G. (2001a). mDia mediates Rho-regulated formation and orientation of stable microtubules. Nat Cell Biol 3, 723-729.
39. Palazzo, A. F., Joseph, H. L., Chen, Y. J., Dujardin, D. L., Alberts, A. S., Pfister, K. K., Vallee, R. B., and Gundersen, G. G. (2001b). Cdc42, dynein, and dynactin regulate MTOC reorientation independent of Rho-regulated microtubule stabilization. Curr Biol 11, 1536-1541.
40. Ponti, A., Machacek, M., Gupton, S. L., Waterman-Storer, C. M., and Danuser, G. (2004). Two distinct actin networks drive the protrusion of migrating cells. Science 305, 1782-1786.
41. Prigozhina, N. L., and Waterman-Storer, C. M. (2004). Protein kinase D-mediated anterograde membrane trafficking is required for fibroblast motility. Curr Biol 14, 88-98.
42. Robinson, J. T., Wojcik, E. J., Sanders, M. A., McGrail, M., and Hays, T. S. (1999). Cytoplasmic dynein is required for the nuclear attachment and migration of centrosomes during mitosis in Drosophila. J Cell Biol 146, 597-608.
43. Royou, A., Sullivan, W., and Karess, R. (2002). Cortical recruitment of nonmuscle myosin II in early syncytial Drosophila embryos: its role in nuclear axial expansion and its regulation by Cdc2 activity. J Cell Biol 158, 127-137.
44. Sakurada, K., Seto, M., and Sasaki, Y. (1998). Dynamics of myosin light chain phosphorylation at Ser19 and Thr18/Ser19 in smooth muscle cells in culture. Am J Physiol 274, C1563-1572.
45. Salmon, W. C., Adams, M. C., and Waterman-Storer, C. M. (2002). Dual-wavelength fluorescent speckle microscopy reveals coupling of microtubule and actin movements in migrating cells. J Cell Biol 158, 31-37.
46. Sander, E. E., van Delft, S., ten Klooster, J. P., Reid, T., van der Kammen, R. A., Michiels, F., and Collard, J. G. (1998). Matrix-dependent Tiam1/Rac Signaling in Epithelial Cells Promotes Either Cell-Cell Adhesion or Cell Migration and Is Regulated by Phosphatidylinositol 3-Kinase. J Cell Biol 143, 1385-1398.
47. Schliwa, M., and Honer, B. (1993). Microtubules, centrosomes and intermediate filaments in directed cell movement. Trends Cell Biol 3, 377-380.
48. Shu, T., Ayala, R., Nguyen, M.-D., Xie, Z., Gleeson, J. G., and Tsai, L.-H. (2004). Ndell Operates in a Common Pathway with LIS1 and Cytoplasmic Dynein to Regulate Cortical Neuronal Positioning. Neuron 44, 263-277.
49. Skop, A. R., and White, J. G. (1998). The dynactin complex is required for cleavage plane specification in early Caenorhabditis elegans embryos. Curr Biol 8, 1110-1116.
50. Soh, J. W., Lee, E. H., Prywes, R., and Weinstein, I. B. (1999). Novel roles of specific isoforms of protein kinase C in activation of the c-fos serum response element. Mol Cell Biol 19, 1313-1324.
51. Solecki, D. J., Model, L., Gaetz, J., Kapoor, T. M., and Hatten, M. E. (2004). Par6a signaling controls glial-guided neuronal migration. Nat Neurosci 7, 1195-1203.
52. Starr, D. A., and Han, M. (2003). ANChors away: an actin based mechanism of nuclear positioning. J Cell Sci 116, 211-216.
53. Stowers, L., Yelon, D., Berg, L. J., and Chant, J. (1995). Regulation of the polarization of T cells toward antigen-presenting cells by Ras-related GTPase CDC42. Proc Natl Acad Sci USA 92, 5027-5031.
54. Tan, I., Ng, C. H., Lim, L., and Leung, T. (2001a). Phosphorylation of a novel myosin binding subunit of protein phosphatase 1 reveals a conserved mechanism in the regulation of actin cytoskeleton. J Biol Chem 276, 21209-21216.
55. Tanaka, T., Serneo, F. F., Higgins, C., Gambello, M. J., Wynshaw-Boris, A., and Gleeson, J. G. (2004). Lis1 and doublecortin function with dynein to mediate coupling of the nucleus to the centrosome in neuronal migration. J Cell Biol 165, 709-721.
56. Tzima, E., Kiosses, W. B., del Pozo, M. A., and Schwartz, M. A. (2003). Localized cdc42 activation, detected using a novel assay, mediates microtubule organizing center positioning in endothelial cells in response to fluid shear stress. J Biol Chem 278, 31020-31023.
57. Von Dassow, G., and Schubiger, G. (1994). How an actin network might cause fountain streaming and nuclear migration in the syncytial Drosophila embryo [published erratum appears in J Cell Biol 1995 September; 130(5):1231-4]. J Cell Biol 127, 1637-1653.
58. Waterman-Storer, C. M., and Salmon, E. D. (1997). Actomyosin-based Retrograde Flow of Microtubules in the Lamella of Migrating Epithelial Cells Influences Microtubule Dynamic Instability and Turnover and Is Associated with Microtubule Breakage and Treadmilling. J Cell Biol 139, 417-434.
59. Wittmann, T., Bokoch, G. M., and Waterman-Storer, C. M. (2003). Regulation of leading edge microtubule and actin dynamics downstream of Rac1. J Cell Biol 161, 845-851.
60. Xiang, X., Beckwith, S., and Morris, N. (1994). Cytoplasmic Dynein is Involved in Nuclear Migration in Aspergillus nidulans. Proc Natl Acad Sci USA 91, 2100-2104.
61. Zenke, F. T., King, C. C., Bohl, B. P., and Bokoch, G. M. (1999). Identification of a Central Phosphorylation Site in p21-activated Kinase Regulating Autoinhibition and Kinase Activity. J Biol Chem 274, 32565-32573.
62. Tan, I., et al., “Intermolecular and intramolecular interactions regulate catalytic activity of myotonic dystrophy kinase-related Cdc42-binding kinase alpha,” Mol. Cell. Biol. 18, 130-140 (2001b).
63. Sumi, T., et al., “Activation of LIM kinases by myotonic dystrophy kinase-related Cdc42-binding kinase alpha,” J. Biol. Chem. 276, 21209-21216 (2001).

1. A composition comprising a pharmaceutically acceptable carrier and a nucleic acid which, when introduced into a cell wherein MRCK is expressed, inhibits the expression of MRCK in the cell.

2. The composition of claim 1, wherein the nucleic acid is siRNA.

3. The composition of claim 1, wherein the nucleic acid is an anti-sense molecule.

4. The composition of claim 1, wherein the nucleic acid is a DNAzyme.

5. The composition of claim 1, wherein the nucleic acid is a ribozyme.

6. The composition of claim 1, wherein the MRCK is human MRCK.

7. The composition of claim 1, wherein the MRCK is encoded by the nucleic acid sequence set forth in SEQ. ID. NO:1.

8. A method for identifying an agent that inhibits MRCK expression in a cell comprising:
(a) contacting the cell with the agent under conditions which would permit the cell to express MRCK in the absence of the agent;
(b) after a suitable period of time, determining the level of MRCK expression in the cell; and
(c) comparing the level of MRCK expression determined in step (b) with the level of MRCK expression determined in the cell in the absence of the agent, whereby a lower amount of expression in the presence of the agent indicates that the agent inhibits MRCK expression in the cell.

9. The method of claim 8, wherein the agent is a nucleic acid.

10. The method of claim 8, wherein the agent is a polypeptide.

11. The method of claim 8, wherein the MRCK is human MRCK.

12. The method of claim 8, wherein the MRCK is encoded by the nucleic acid sequence set forth in SEQ. ID. NO:1.

13. A method for identifying an agent that inhibits MRCK-mediated kinase activity in a cell comprising:
(a) contacting the cell with the agent under conditions which would permit the cell to express MRCK in the absence of the agent;
(b) after a suitable period of time, determining the level of MRCK-mediated kinase activity in the cell; and
(c) comparing the level of MRCK-mediated kinase activity determined in step (b) with the MRCK-mediated kinase activity determined in the cell in the absence of the agent, whereby a lower amount of activity in the presence of the agent indicates that the agent inhibits MRCK-mediated kinase activity in the cell.

14. The method of claim 13, wherein the agent is a nucleic acid.

15. The method of claim 13, wherein the agent is a polypeptide.

16. The method of claim 13, wherein the MRCK is human MRCK.

17. The method of claim 13, wherein the MRCK is encoded by the nucleic acid sequence set forth in SEQ. ID. NO:1.

18-31. (canceled)


SEQUENCE LISTING (6 sequences)

SEQ ID NO: 1 - DNA, 8019 bp, Homo sapiens
gcggcccggtgcgggtgtcggggagaccgggctctctgcccggcgcggcgcggcgcggctcggcccacgagcgaccaccgacatggagtgggctcgggcggccaagtagccgcttctccggagcccggtgccagtgccgcccgcagcccgccttccacccccggccgcgccgccggtcaggccctagggtgaagccgggaggaaaatgaagagttttcaccggaatccgttgaaaataggactgactgcaaagccttaaagaaagaaggacctcgggaggagaaacgaaaagccgcctccgggcaagacttggcgtgctccgagccgaggggctgcttcagggacctcgccccctccctttcccgctggagaaattgccgctgatgcattatccaagtggtggttgggaggatttgcagcaacatttttggttttccctcccccttctatgcattctgtttttttcctcccttttctgtttttcttcttcccgggaagtgaattgctgatgcaaatcggactttattcattaatgatgcaaccggattcgtttcaggattacgttgcacgagttgaattttgaatgaaggagaagagtttttttttttttttttaaagaagtgttgactctctagttcgttgtacttttaattattattttatttaaatatacgacttaattgtattcttttaaaaatgcattaagtatatattttatggtaatttaccctcaaaatatatgtatatgggtgaaattgaagacgcttcagttaagtgaggttactggtgtgttggatgtttaattcagcaccagcattgcatgacagttgtttgaataacaagtggtttatttttaaaaccataccttttaaaatttaggttcagataatagtaaaagtcatcataataatttaaaggaaaaccagcagaaatcgaagcaaacatgtctggagaagtgcgtttgaggcagttggagcagtttattttggacgggcccgctcagaccaatgggcagtgcttcagtgtggagacattactggatatactcatctgcctttatgatgaatgcaataattctccattgagaagagagaagaacattctcgaatacctagaatgggctaaaccatttacttctaaagtgaaacaaatgcgattacatagagaagactttgaaatattaaaggtgattggtcgaggagcttttggggaggttgctgtagtaaaactaaaaaatgcagataaagtgtttgccatgaaaatattgaataaatgggaaatgctgaaaagagctgagacagcatgttttcgtgaagaaagggatgtattagtgaatggagacaataaatggattacaaccttgcactatgctttccaggatgacaataacttatacctggttatggattattatgttggtggggatttgcttactctactcagcaaatttgaagatagattgcctgaagatatggctagattttacttggctgagatggtgatagcaattgactcagttcatcagctacattatgtacacagagacattaaacctgacaatatactgatggatatgaatggacatattcggttagcagattttggttcttgtctgaagctgatggaagatggaacggttcagtcctcagtggctgtaggaactccagattatatctctcctgaaatccttcaagccatggaagatggaaaagggagatatggacctgaatgtgactggtggtctttgggggtctgtatgtatgaaatgctttacggagaaacaccattttatgcagaatcgctggtggagacatacggaaaaatcatgaaccacaaagagaggtttcagtttccagcccaagtgactgatgtgtctgaaaatgctaaggatcttattcgaaggctcatttgtagcagagaacatcgacttggtcaaaatggaatagaagactttaagaaacacccatttttcagtggaattgattgggataatattcggaactgtgaagcaccttatattccagaagttagtagcccaacagatacatcgaattttgatgtagatgatgattgtttaaaaaattctgaaacgatgcccccaccaacacatactgcattttctggccaccatctgccatttgttggttttacatatactagtagctgtgtactttctgatcggagctgtttaagagttacggctggtcccacctcactggatcttgatgttaatgttcagaggactctagacaacaacttagcaactgaagcttatgaaagaagaattaagcgccttgagcaagaaaaacttgaactcagtagaaaacttcaagagtcaacacagactgtccaagctctgcagtattcaactgttgatggtccactaacagcaagcaaagatttagaaataaaaaacttaaaagaagaaattgaaaaactaagaaaacaagtaacagaatcaagtcatttggaacagcaacttgaagaagctaatgctgtgaggcaagaactagatgatgcttttagacaaatcaaggcttatgaaaaacaaatcaaaacgttacaacaagaaagagaagatctaaataaggaactagtccaggctagtgagcgattaaaaaaccaatccaaagagctgaaagacgcacactgtcagaggaaactggccatgcaggaattcatggagatcaatgagcggctaacagaattgcacacccaaaaacagaaacttgctcgccatgtccgagataaggaagaagaggtggacctggtgatgcaaaaagttgaaagcttaaggcaagaactgcgcagaacagaaagagccaaaaaagagctggaagttcatacagaagctctagctgctgaagcatctaaagacaggaagctacgtgaacagagtgagcactattctaagcaactggaaaatgaattggagggactgaagcaaaaacaaattagttactcaccaggagtatgcagcatagaacatcagcaagagataaccaaactaaagactgatttggaaaagaaaagtatcttttatgaagaagaattatctaaaagagaaggaatacatgcaaatgaaataaaaaatcttaagaaagaactgcatgattcagaaggtcagcaacttgctctcaacaaagaaattatgattttaaaagacaaattggaaaaaaccagaagagaaagtcaaagtgaaagggaggaatttgaaagtgagttcaaacaacaatatgaacgagaaaaagtgttgttaactgaagaaaataaaaagctgacgagtgaacttgataagcttactactttgtatgagaacttaagtatacacaaccagcagttagaagaagaggttaaagatctagcagacaagaaagaatcagttgcacattgggaagcccaaatcacagaaataattcagtgggtcagcgatgaaaaggatgcacgagggtatcttcaggccttagcttctaaaatgactgaagaattggaggcattaagaaattccagcttgggtacacgagcaacagatatgccctggaaaatgcgtcgttttgcgaaactggatatgtcagctagactggagttgcagtcggctctggatgcagaaataagagccaaacaggccatccaagaagagttgaataaagttaaagcatctaatatcataacagaatgtaaactaaaagattcagagaagaagaacttggaactactctcagaaatcgaacagctgataaaggacactgaagagcttagatctgaaaagggtatagagcaccaagactcacagcattctttcttggcatttttgaatacgcctaccgatgctctggatcaatttgaaactgtagactccactccactttcagttcacacaccaaccttaaggaaaaaaggatgtcctggttcaactggctttccacctaagcgcaagactcaccagttttttgtaaaatcttttactactcctaccaagtgtcatcagtgtacctccttgatggtgggtttaataagacagggctgttcatgtgaagtgtgtggattctcatgccatataacttgtgtaaacaaagctccaaccacttgtccagttcctcctgaacagacaaaaggtcccctgggtatagatcctcagaaaggaataggaacagcatatgaaggtcatgtcaggattcctaagccagctggagtgaagaaagggtggcagagagcactggctatagtgtgtgacttcaaactctttctgtacgatattgctgaaggaaaagcatctcagcccagtgttgtcattagtcaagtgattgacatgagggatgaagaattttctgtgagttcagtcttggcttctgatgttatccatgcaagtcggaaagatataccctgtatatttagggtcacagcttcccagctctcagcatctaataacaaatgttcaatcctgatgctagcagacactgagaatgagaagaataagtgggtgggagtgctgagtgaattgcacaagattttgaagaaaaacaaattcagagaccgctcagtctatgttcccaaagaggcttatgacagcactctacccctcattaaaacaacccaggcagccgcaatcatagatcatgaaagaattgctttgggaaacgaagaagggttatttgttgtacatgtcaccaaagatgaaattattagagttggtgacaataagaagattcatcagattgaactcattccaaatgatcagcttgttgctgtgatctcaggacgaaatcgtcatgtacgactttttcctatgtcagcattggatgggcgagagaccgatttttacaagctgtcagaaactaaagggtgtcaaaccgtaacttctggaaaggtgcgccatggagctctcacatgcctgtgtgtggctatgaaaaggcaggtcctctgttatgaactatttcagagcaagacccgtcacagaaaatttaaagaaattcaagtcccatataatgtccagtggatggcaatcttcagtgaacaactctgtgtgggattccagtcaggatttctaagataccccttgaatggagaaggaaatccatacagtatgctccattcaaatgaccatacactatcatttattgcacatcaaccaatggatgctatctgcgcagttgagatctccagtaaagaatatctgctgtgttttaacagcattgggatatacactgactgccagggccgaagatctagacaacaggaattgatgtggccagcaaatccttcctcttgttgttacaatgcaccatatctctcggtgtacagtgaaaatgcagttgatatctttgatgtgaactccatggaatggattcagactcttcctctcaaaaaggttcgacccttaaacaatgaaggatcattaaatcttttagggttggagaccattagattaatatatttcaaaaataagatggcagaaggggacgaactggtagtacctgaaacatcagataatagtcggaaacaaatggttagaaacattaacaataagcggcgttattccttcagagtcccagaagaggaaaggatgcagcagaggagggaaatgctacgagatccagaaatgagaaataaattaatttctaatccaactaattttaatcacatagcacacatgggtcctggagatggaatacagatcctgaaagatctgcccatgaaccctcggcctcaggaaagtcggacagtattcagtggctcagtcagtattccatctatcaccaaatcccgccctgagccaggccgctccatgagtgctagcagtggcttgtcagcaaggtcatccgcacagaatggcagcgcattaaagagggaattctctggaggaagctacagtgccaagcggcagcccatgccctccccgtcagagggctctttgtcctctggaggcatggaccaaggaagtgatgccccagcgagggactttgacggagaggactctgactctccgaggcattccacagcttccaacagttccaacctaagcagccccccaagcccagcttcaccccgaaaaaccaagagcctctccctggagagcactgaccgcgggagctgggacccgtgagctgcctcagcactgggacctctcgctctccgctccctgccactcgcctcctctcactttcatctcttccctccacctcgcctgctcggcctgaaagccaccaggggctggcagcagtagcaggacagggattcaggagttctgacgacacgactctcagatccacgcccccagcctaacagcaacaacaaagacagactttccgtagcagcttagattaacgttgatttcattccatgcacttagagttgctttcagtaacattttacccctactcccaaaggtagcttaaatagacagattacacaaatgtaagtgataagaataagattagacagattttgctttcacagtagagtctcattatagtcctaaaatagctcatgggcttctccgcatccagaagggagaattggtccctggagtggctcactaagctcttaatcagcaaacgcagtgagtatcaacctgattgttgccaggaaatccttatgaattaaaacaatgcatattttactacagtacagagtttaaatgaatacataaatgtagaagtactgaatgtatatatttaaaaggagcctcttgtattcaacaaaagatggatgcatatataagagagatgatttaatttaaagaaatatgttgtttcttgtctgtaatgtaatgtaaagggtggaaaggcctcaagctcacatttgtagagagagagcgagagaaatcagagttccctttattgccctgtcctcaaactggtcataggctctagtcacctggggagctgtagaaaacacttgcagagccaggttttgctggtttggggcatgccctgggcaccagagctttaacatttgaagccacttcagcagcagcagcaaaaggcgaactcatctctacccaagatgtttcttttcctagtggtggaatttgaacacttctcactttttattgtattttatcttccgcagataaatgtagaaatacacgattctgtcacctctgatcccttccatctgaaaggttacaaggagtgttgtagcttctgaaggtgcagaaaacaatttctaaaaatgcttttattcctgggctaatcctgtccctccctaagtcacagcgaggtgtctgtcccagggctggagatgcttcccaaggaggagtctgttttgttgagagtgggcgtgggcttcttcacataagcctggggaaggaagaaaaaacggctttcattaccaaataatgtaaaacctcaaaagcaagggcttcaacagccttaaccaaatattattccccatagccagtggaaaatggatgtgacaaccccagtgcgcaggccagagtgagtgagcccagcacggcgctccgactggcttcctctctcaggtgctggattgtggggttagtggcatttccagctggattcctcctgttgtagttgccataaggaaatgagatgcagaatcagaaggatctatttctacagaatcatttcaccagttaagcacatgagtagagaaagagataaaaataaaagtatctcatgaaggaaagagattttgcctctcttttacttttcacctaagtttctctgagaaatagagacaggattctctctttaaaattcagtgaaaatgaagaaagttttcctgcagttgctaacctgagttgcagtgtttaaggccatcatttcactgctgctgtctgtgactccacgtctgtgtcactgaggtgacctgcgtgtcactgaggtggccaccatgctggcctgcggcatgtgcagggagctgaggctgtttccaggtgatgctgctgtgtggagaaggttctgagatgcagtgagggaagaaaggatcctgctggggattccattgtaagcacctataatcgggaattttcatgtaacagctttgacatttaaa

SEQ ID NO: 2 - protein, 1719 aa, Homo sapiens
MSGEVRLRQLEQFILDGPAQTNGQCFSVETLLDILICLYDECNNSPLRREKNILEYLEWAKPFTSKVKQMRLHREDFEILKVIGRGAFGEVAVVKLKNADKVFAMKILNKWEMLKRAETACFREERDVLVNGDNKWITTLHYAFQDDNNLYLVMDYYVGGDLLTLLSKFEDRLPEDMARFYLAEMVIAIDSVHQLHYVHRDIKPDNILMDMNGHIRLADFGSCLKLMEDGTVQSSVAVGTPDYISPEILQAMEDGKGRYGPECDWWSLGVCMYEMLYGETPFYAESLVETYGKIMNHKERFQFPAQVTDVSENAKDLIRRLICSREHRLGQNGIEDFKKHPFFSGIDWDNIRNCEAPYIPEVSSPTDTSNFDVDDDCLKNSETMPPPTHTAFSGHHLPFVGFTYTSSCVLSDRSCLRVTAGPTSLDLDVNVQRTLDNNLATEAYERRIKRLEQEKLELSRKLQESTQTVQALQYSTVDGPLTASKDLEIKNLKEEIEKLRKQVTESSHLEQQLEEANAVRQELDDAFRQIKAYEKQIKTLQQEREDLNKELVQASERLKNQSKELKDAHCQRKLAMQEFMEINERLTELHTQKQKLARHVRDKEEEVDLVMQKVESLRQELRRTERAKKELEVHTEALAAEASKDRKLREQSEHYSKQLENELEGLKQKQISYSPGVCSIEHQQEITKLKTDLEKKSIFYEEELSKREGIHANEIKNLKKELHDSEGQQLALNKEIMILKDKLEKTRRESQSEREEFESEFKQQYEREKVLLTEENKKLTSELDKLTTLYENLSIHNQQLEEEVKDLADKKESVAHWEAQITEIIQWVSDEKDARGYLQALASKMTEELEALRNSSLGTRATDMPWKMRRFAKLDMSARLELQSALDAEIRAKQAIQEELNKVKASNIITECKLKDSEKKNLELLSEIEQLIKDTEELRSEKGIEHQDSQHSFLAFLNTPTDALDQFETVDSTPLSVHTPTLRKKGCPGSTGFPPKRKTHQFFVKSFTTPTKCHQCTSLMVGLIRQGCSCEVCGFSCHITCVNKAPTTCPVPPEQTKGPLGIDPQKGIGTAYEGHVRIPKPAGVKKGWQRALAIVCDFKLFLYDIAEGKASQPSVVISQVIDMRDEEFSVSSVLASDVIHASRKDIPCIFRVTASQLSASNNKCSILMLADTENEKNKWVGVLSELHKILKKNKFRDRSVYVPKEAYDSTLPLIKTTQAAAIIDHERIALGNEEGLFVVHVTKDEIIRVGDNKKIHQIELIPNDQLVAVISGRNRHVRLFPMSALDGRETDFYKLSETKGCQTVTSGKVRHGALTCLCVAMKRQVLCYELFQSKTRHRKFKEIQVPYNVQWMAIFSEQLCVGFQSGFLRYPLNGEGNPYSMLHSNDHTLSFIAHQPMDAICAVEISSKEYLLCFNSIGIYTDCQGRRSRQQELMWPANPSSCCYNAPYLSVYSENAVDIFDVNSMEWIQTLPLKKVRPLNNEGSLNLLGLETIRLIYFKNKMAEGDELVVPETSDNSRKQMVRNINNKRRYSFRVPEEERMQQRREMLRDPEMRNKLISNPTNFNHIAHMGPGDGIQILKDLPMNPRPQESRTVFSGSVSIPSITKSRPEPGRSMSASSGLSARSSAQNGSALKREFSGGSYSAKRQPMPSPSEGSLSSGGMDQGSDAPARDFDGEDSDSPRHSTASNSSNLSSPPSPASPRKTKSLSLESTDRGSWDP

SEQ ID NO: 3 - DNA, 6782 bp, Homo sapiens
gggcggggctgagggcggcgggggcgggccgcccgagctgggagggcggcggcgccgaggggaggagagcggcccatggacccgcggggcccggcgccccagactctgcgccgtcgggacggagcccaagatgtcggcctaggccggggcgcgacgacgcggacggggcggcgaggaggcgccgctgctgccggggctcgcagccgccgagcccccgagggcgcgccctgacggactggccgagccggcggtgagaggccggcgcgtcgggagcgggccgcgcggcaccatgtcggccaaggtgcggctcaagaagctggagcagctgctcctggacgggccctggcgcaacgagagcgccctgagcgtggaaacgctgctcgacgtgctcgtctgcctgtacaccgagtgcagccactcggccctgcgccgcgacaagtacgtggccgagttcctcgagtgggctaaaccatttacacagctggtgaaagaaatgcagcttcatcgagaagactttgaaataattaaagtaattggaagaggtgcttttggtgaggttgctgttgtcaaaatgaagaatactgaacgaatttatgcaatgaaaatcctcaacaagtgggagatgctgaaaagagcagagaccgcgtgcttccgagaggagcgcgatgtgctggtgaacggcgactgccagtggatcaccgcgctgcactacgcctttcaggacgagaaccacctgtacttagtcatggattactatgtgggtggtgatttactgaccctgctcagcaaatttgaagacaagcttccggaagatatggcgaggttctacattggtgaaatggtgctggccattgactccatccatcagcttcattacgtgcacagagacattaaacctgacaatgtccttttggacgtgaatggtcatatccgcctggctgactttggatcatgtttgaagatgaatgatgatggcactgtgcagtcctccgtggccgtgggcacacctgactacatctcgccggagatcctgcaggcgatggaggacggcatgggcaaatacgggcctgagtgtgactggtggtctctgggtgtctgcatgtatgagatgctctatggagaaacgccgttttatgcggagtcactcgtggagacctatgggaagatcatgaaccatgaagagcgattccagttcccatcccatgtcacggatgtatctgaagaagcgaaggacctcatccagagactgatctgcagtagagaacgccggctggggcagaatggaatagaggatttcaaaaagcatgcgttttttgaaggtctaaattgggaaaatatacgaaacctagaagcaccttatattcctgatgtgagcagtccctctgacacatccaacttcgacgtggatgacgacgtgctgagaaacacggaaatattacctcctggttctcacacaggcttttctggattacatttgccattcattggttttacattcacaacggaaagctgtttttctgatcgaggctctctgaagagcataatgcagtccaacacattaaccaaagatgaggatgtgcagcgggacctggagcacagcctgcagatggaagcttacgagaggaggattcggaggctggaacaggagaagctggagctgagcaggaagctgcaagagtccacccagaccgtgcagtccctccacggctcatctcgggccctcagcaattcaaaccgagataaagaaatcaaaaagctaaatgaagaaatcgaacgcttgaagaataaaatagcagattcaaacaggctcgagcgacagcttgaggacacagtggcgcttcgccaagagcgtgaggactccacgcagcggctgcgggggctggagaagcagcaccgcgtggtccggcaggagaaggaggagctgcacaagcaactggttgaagcctcagagcggttgaaatcccaggccaaggaactcaaagatgcccatcagcagcgaaagctggccctgcaggagttctcggagctgaacgagcgcatggcagagctccgtgcccagaagcagaaggtgtcccggcagctgcgagacaaggaggaggagatggaggtggccacgcagaaggtggacgccatgcggcaggaaatgcggagagctgagaagctcaggaaagagctggaagctcagcttgatgatgctgttgctgaggcctccaaggagcgcaagcttcgtgagcacagcgagaacttctgcaagcaaatggaaagcgagctggaggccctcaaggtgaagcaaggaggccggggagcgggtgccaccttagagcaccagcaagagatttccaaaatcaaatccgagctggagaagaaagtcttattttatgaagaggaattggtcagacgtgaggcctcccatgtgctagaagtgaaaaatgtgaagaaggaggtgcatgattcagaaagccaccagctggccctgcagaaagaaatcttgatgttaaaagataagttagaaaagtcaaagcgagaacggcataacgagatggaggaggcagtaggtacaataaaagataaatacgaacgagaaagagcgatgctgtttgatgaaaacaagaagctaactgctgaaaatgaaaagctctgttcctttgtggataaactcacagctcaaaatagacagctggaggatgagctgcaggatctggcagccaagaaggagtcagtggcccactgggaagctcagattgcggaaatcattcagtgggtcagtgacgagaaagatgcccggggttaccttcaagctcttgcttccaagatgaccgaagagctcgaggctttgaggagttctagtctggggtcaagaacactggacccgctgtggaaggtgcgccgcagccagaagctggacatgtccgcgcggctggagctgcagtcggccctggaggcggagatccgggccaagcagcttgtccaggaggagctcaggaaggtcaaggacgccaacctcaccttggaaagcaaactaaaggattccgaagccaaaaacagagaattattagaagaaatggaaattttgaagaaaaagatggaagaaaaattcagagcagatactgggctcaaacttccagattttcaggattccatttttgagtatttcaacactgctcctcttgcacatgacctgacatttagaaccagctcagctagtgagcaagaaacacaagctccgaagccagaagcgtccccgtcgatgtctgtggctgcatcagagcagcaggaggacatggctcggcccccgcagaggccatccgctgtgccgttgcccaccacgcaggccctggctctggctggaccgaagccaaaagctcaccagttcagcatcaagtccttctccagccctactcagtgcagccactgcacctccctgatggttgggctgatccggcagggctacgcctgcgaggtgtgttcctttgcttgccacgtgtcctgcaaagacggtgccccccaggtgtgcccaatacctcccgagcagtccaagaggcctctgggcgtggacgtgcagcgaggcatcggaacagcctacaaaggccatgtcaaggtcccaaagcccacgggggtgaagaagggatggcagcgcgcatatgcagtcgtctgtgagtgcaagctcttcctgtatgatctgcctgaaggaaaatccacccagcctggtgtcattgcgagccaagtcttggatctcagagatgacgagttttccgtgagctcagtcctggcctcagatgtcattcatgctacacgccgagatattccatgtatattcagggtgacggcctctctcttaggtgcaccttctaagaccagctcgctgctcattctgacagaaaatgagaatgaaaagaggaagtgggttgggattctagaaggactccagtccatccttcataaaaaccggctgaggaatcaggtcgtgcatgttcccttggaagcctacgacagctcgctgcctctcatcaaggccatcctgacagctgccatcgtggatgcagacaggattgcagtcggcctagaagaagggctctatgtcatagaggtcacccgagatgtgatcgtccgtgccgctgactgtaagaaggtacaccagatcgagcttgctcccagggagaagatcgtaatcctcctctgtggccggaaccaccatgtgcacctctatccgtggtcgtcccttgatggagcggaaggcagctttgacatcaagcttccggaaaccaaaggctgccagctcatggccacggccacactcaagaggaactctggcacctgcctgtttgtggccgtgaaacggctgatcctttgctatgagatccagagaacgaagccattccacagaaagttcaatgagattgtggctcccggcagcgtgcagtgcctggcggtgctcagggacaggctctgtgtgggctacccttctgggttctgcctgctgagcatccagggggacgggcagcctctaaacctggtaaatcccaatgacccctcgcttgcgttcctctcacaacagtcttttgatgccctttgtgctgtggagctcgaaagcgaggagtacctgctttgcttcagccacatgggactgtacgtggacccgcaaggccggagggcacgcgcgcaggagctcatgtggcctgcggctcctgtcgcctgtagttgcagccccacccacgtcacggtgtacagcgagtatggcgtggacgtctttgatgtgcgcaccatggagtgggtgcagaccatcggcctgcggaggataaggcccctgaactctgaaggcaccctcaacctcctcaactgcgagcctccacgcttgatctacttcaagagcaagttctcgggagcggttctcaacgtgccggacacctccgacaacagcaagaagcagatgctgcgcaccaggagcaaaaggcggttcgtcttcaaggtcccagaggaagagagactgcagcagaggcgagagatgcttagagacccagaattgagatccaaaatgatatccaacccaaccaacttcaaccacgtggcccacatgggcccaggcgacggcatgcaggtgctcatggacctgcctctgagtgctgtgcccccctcccaggaggaaaggccgggccccgctcccaccaacctggctcgccagcctccatccaggaacaagccctacatctcgtggccctcatcaggtggatcggagcctagcgtgactgtgcctctgagaagtatgtctgatccagaccaggactttgacaaagagcctgattcggactccaccaaacactcaactccatcgaatagctccaaccccagcggcccaccgagccccaactccccccacaggagccagctccccctcgaaggcctggagcagccggcctgtgacacctgaagccgccagctcgccacaggggccagggagctggagatggcctccagcgtcagtgccaagactgagcgggccctccagtgttgtccaaggaaatgtagaatcactttgtagatatggagatgaagaagacaaatctttattataatattgatcagttttatgccgcattgttcgtggcagtagaccacatctgttcgtctgcacagctgtgaggcgatgctgttccatctgcacatgaaggacccccatacagcctgtctcccacccctgacaacccgagagggcatatggggccctgccaacaccacttcctcagcagaaacccgtcatgacgcggctgcttcggaagcagacatctggggacacagcctcagtacccagtcttttccctagttcctgaaactttcctaggaccttaagagaatagtaggaggtcctatagcattcccagtgtcactagaattttgaagacaggaaagtggaggttagtctgtggcctttttttcatttagccattgcacagtcagctgcagaagtcctgctgaccacctagtcatggacaaaggcccaggaccagtgacaccctgcgtccctgtgtgcattaagttcattctgggtcgcagccatgaagtgtcaccagtatctactactgtgaagtcagctgtgctgttttccattcgcttccacggcttctgcctcctgccataaaaccagcgagtgtcgtggtgcaggcaggccctgtggcctgctgggctgagggaagtcagagccccagggcgccacgaagcagccactgggataccccaccccgccccgccctgcccccccccccccccaccagtcctgcccccgcatggagcccccgtgattagtagcccgtatgatcacgtagacccacccaacacactcctgcacactggccccggcccacggcacagcaatcccctgcgcgtggatttcacctcaccctttgtaccagatgttgagtgaccagctctgtggccctgtgtcgtcagaggcttgtgattaactgtggcggcagacacagcttgtccacagcttgggccaggcttcccctgtcctcccaccggtcggctgcttggcaaggctgttcaggacgtgcacttccccaagtcggcactgagtggcccagcaccgcctagccctgccaccccactgccctcctgggccttctgctggatgggcacctggggggttctggtttttacttttttaatgtaagtctcagtctttgtaattaattattgaattgtgagaacatttttgaacaatttacctgtcaataaagcagaagacggcagttttaaagttaaaaaaaaaaaaaaaaaaaaaaaaa

SEQ ID NO: 4 - protein, 1711 aa, Homo sapiens
MSAKVRLKKLEQLLLDGPWRNESALSVETLLDVLVCLYTECSHSALRRDKYVAEFLEWAKPFTQLVKEMQLHREDFEIIKVIGRGAFGEVAVVKMKNTERIYAMKILNKWEMLKRAETACFREERDVLVNGDCQWITALHYAFQDENHLYLVMDYYVGGDLLTLLSKFEDKLPEDMARFYIGEMVLAIDSIHQLHYVHRDIKPDNVLLDVNGHIRLADFGSCLKMNDDGTVQSSVAVGTPDYISPEILQAMEDGMGKYGPECDWWSLGVCMYEMLYGETPFYAESLVETYGKIMNHEERFQFPSHVTDVSEEAKDLIQRLICSRERRLGQNGIEDFKKHAFFEGLNWENIRNLEAPYIPDVSSPSDTSNFDVDDDVLRNTEILPPGSHTGFSGLHLPFIGFTFTTESCFSDRGSLKSIMQSNTLTKDEDVQRDLEHSLQMEAYERRIRRLEQEKLELSRKLQESTQTVQSLHGSSRALSNSNRDKEIKKLNEEIERLKNKIADSNRLERQLEDTVALRQEREDSTQRLRGLEKQHRVVRQEKEELHKQLVEASERLKSQAKELKDAHQQRKLALQEFSELNERMAELRAQKQKVSRQLRDKEEEMEVATQKVDAMRQEMRRAEKLRKELEAQLDDAVAEASKERKLREHSENFCKQMESELEALKVKQGGRGAGATLEHQQEISKIKSELEKKVLFYEEELVRREASHVLEVKNVKKEVHDSESHQLALQKEILMLKDKLEKSKRERHNEMEEAVGTIKDKYERERAMLFDENKKLTAENEKLCSFVDKLTAQNRQLEDELQDLAAKKESVAHWEAQIAEIIQWVSDEKDARGYLQALASKMTEELEALRSSSLGSRTLDPLWKVRRSQKLDMSARLELQSALEAEIRAKQLVQEELRKVKDANLTLESKLKDSEAKNRELLEEMEILKKKMEEKFRADTGLKLPDFQDSIFEYFNTAPLAHDLTFRTSSASEQETQAPKPEASPSMSVAASEQQEDMARPPQRPSAVPLPTTQALALAGPKPKAHQFSIKSFSSPTQCSHCTSLMVGLIRQGYACEVCSFACHVSCKDGAPQVCPIPPEQSKRPLGVDVQRGIGTAYKGHVKVPKPTGVKKGWQRAYAVVCECKLFLYDLPEGKSTQPGVIASQVLDLRDDEFSVSSVLASDVIHATRRDIPCIFRVTASLLGAPSKTSSLLILTENENEKRKWVGILEGLQSILHKNRLRNQVVHVPLEAYDSSLPLIKAILTAAIVDADRIAVGLEEGLYVIEVTRDVIVRAADCKKVHQIELAPREKIVILLCGRNHHVHLYPWSSLDGAEGSFDIKLPETKGCQLMATATLKRNSGTCLFVAVKRLILCYEIQRTKPFHRKFNEIVAPGSVQCLAVLRDRLCVGYPSGFCLLSIQGDGQPLNLVNPNDPSLAFLSQQSFDALCAVELESEEYLLCFSHMGLYVDPQGRRARAQELMWPAAPVACSCSPTHVTVYSEYGVDVFDVRTMEWVQTIGLRRIRPLNSEGTLNLLNCEPPRLIYFKSKFSGAVLNVPDTSDNSKKQMLRTRSKRRFVFKVPEEERLQQRREMLRDPELRSKMISNPTNFNHVAHMGPGDGMQVLMDLPLSAVPPSQEERPGPAPTNLARQPPSRNKPYISWPSSGGSEPSVTVPLRSMSDPDQDFDKEPDSDSTKHSTPSNSSNPSGPPSPNSPHRSQLPLEGLEQPACDT

SEQ ID NO: 5 - DNA, 4680 bp, Homo sapiens
atggagcggcggctgcgcgcgctggagcagctggcgcggggcgaggccggcggctgcccggggctcgacggcctcctagatctgctgctggcgctgcaccacgagctcagcagcggccccctacggcgggagcgcagcgtggcgcagttcctgagctgggccagccccttcgtatcaaaggtgaaagaactgcgtctgcagagagatgactttgagatcttgaaggtgatcggccgaggagcctttggggaggtcaccgtggtgaggcagagggacactgggcagatttttgccatgaaaatgctgcacaagtgggagatgctgaagagggctgagacagcctgtttccgggaggagcgggatgtgctcgtgaaaggggacagccgttgggtgaccactctgcactatgccttccaagacgaggagtacctgtaccttgtgatggactactatgctggtggggacctcctgacgctgctgagccgcttcgaggaccgtctcccgcccgagctggcccagttctacctggctgagatggtgctggccatccactcgctgcaccagctgggttatgtccacagggatgtcaagccagacaacgtcctgctggatgtgaacgggcacattcgcctggctgacttcggctcctgcctgcgtctcaacaccaacggcatggtggattcatcagtggcagtagggacgccggactatatctcccctgagatcctgcaggccatggaggagggcaagggccactacggcccacagtgtgactggtggtcgcttggagtctgcgcctatgagctgctctttggggagacgcccttctatgctgagtccttggtggaaacctacggcaagatcatgaaccacgaggaccacctgcagttccccccggacgtgcctgacgtgccagccagcgcccaagacctgatccgccagctgctgtgtcgccaggaagagcggctaggccgtggtgggctggatgacttccggaaccatcctttcttcgaaggcgtggactgggagcggctggcgagcagcacggccccctatattcctgagctgcgggggcccatggacacctccaactttgatgtggatgacgacaccctcaaccatccagggaccctgccaccgccctcccacggggccttctccggccatcacctgccattcgtgggcttcacctacacctcaggcagtcacagtcctgagagcagctctgaggcttgggctgccctggagcggaagctccagtgtctggagcaggagaaggtggagctgagcaggaagcaccaagaggccctgcacgcccccacagaccatcgggagctggagcagctacggaaggaagtgcagactctgcgggacaggctgccagagatgctgagggacaaggcctcattgtcccagacggatgggcccccagctggtagcccaggtcaggacagtgacctacggcaggagcttgaccgacttcaccgggagctggccgagggtcgggcagggctgcaggctcaggagcaggagctctgcagggcccaggggcagcaggaggagctgcttcagaggctacaggaggcccaggagagagaggcggccacagctagccagacccgggccctgagctcccagctggaggaagcccgggctgcccagagggagctggaggcccaggtgtcctccctgagccggcaggtgacgcagctgcagggacagtgggagcaacgccttgaggagtcgtcccaggccaagaccatccacacggcctctgagaccaacgggatgggaccccctgagggtgggcctcaggaggcccaactgaggaaggaggtggccgccctgcgagagcagctggagcaggcccacagccacaggcggagtggtaaggaggaggctctgtgccagctgcaggaggaaaaccggaggctgagccgggagcaggagcggctagaagcagagctggcccaggagcaggagagcaagcagcggctggagggtgagcggcgggagacggagagcaactgggaggcccagctcgccgacatcctcagctgggtgaatgatgagaaggtctcaagaggctacctgcaggccctggccaccaagatggcagaggagctggagtccttgaggaacgtaggcacccagacgctccctgcccggccactggaccaccagtggaaggcgcggcgactgcagaagatggaggcctcggccaggctggagctgcagtcagcgctggaggccgagatccgcgccaagcagggcctgcaggagcggctgacacaggtgcaggaggcccagctgcaggctgagcgccgtctgcaggaggccgagaagcagagccaggccctgcaacaggagctcgccatgctgcgggaggagctgcgggcccgagggccagtggacaccaagccctcaaactccctgattcccttcctgtccttccggagctcagagaaggattctgccaaggaccctggcatctcaggagaggccacaaggcatggaggagagccagatctgaggccggagggccgacgcagcctgcgcatgggggctgtgttccccagagcacccactgccaacacagcctctacagaaggtcttcctgctaagcccggctcacacacgctgcgcccccggagcttcccatccccgaccaagtgtctccgctgcacctcgctgatgctgggcctgggccgccagggcctgggttgtgatgcctgcggctacttttgtcacacaacctgtgccccacaggccccaccctgccccgtgccccctgacctcctccgcacagccctgggagtacaccccgaaacaggcacaggcactgcctatgagggctttctgtcggtgccgcggccctcaggtgtccggcggggctggcagcgcgtgtttgctgccctgagtgactcacgcctgctgctgtttgacgcccctgacctgaggctcagcccgcccagtggggccctcctgcaggtcctagatctgagggacccccagttctcggctacccctgtcctggcctctgatgttatccatgcccaatccagggacctgccacgcatctttagggtgacaacctcccagctggcagtgccgcccaccacgtgcactgtgctgctgctggcagagagcgagggggagcgggaacgctggctgcaggtgctgggtgagctgcagcggctgctgctggacgcgcggccaagaccccggcccgtgtacacactcaaggaggcttacgacaacgggctgccgctgctgcctcacacgctctgcgctgccatcctcgaccaggatcgacttgcgcttggcaccgaggaggggctctttgtcatccatctgcgcagcaacgacatcttccaggtgggggagtgccggcgcgtgcagcagctgaccttgagccccagtgcaggcctgctggtcgtgctgtgtggccgcggccccagcgtgcgtctctttgccctggcggagctggagaacatagaggtagcaggtgccaagatccccgagtctcgaggctgccaggtgctggcagctggaagcatcctgcaggcccgcaccccggtgctctgtgtagccgtcaagcgccaggtgctctgctaccagctgggcccgggccctgggccctggcagcgccgcatccgtgagctgcaggcacctgccactgtgcagagcctggggctgctgggcgaccggctatgtgtgggcgccgccggtggctttgcactctacccgctgctcaacgaggctgcgccgttggcgctgggggccggtttggtgcctgaggagctgccaccatcccgcgggggcctgggtgaggcactgggtgccgtggagcttagcctcagcgagttcctgctactcttcaccactgctggcatctacgtggatggcgcaggccgcaagtctcgtggccacgagctgttgtggccagcagcgcccatgggctgggggtatgcggccccctacctgacagtgttcagcgagaactccatcgatgtgtttgacgtgaggagggcagaatgggtgcagaccgtgccgctcaagaaggtgcggcccctcaatccagagggctccctgttcctctacggcaccgagaaggtccgcctgacctacctcaggaaccagctggcagagaaggacgagttcgacatcccggacctcaccgacaacagccggcgccagctgttccgcaccaagagcaagcgccgcttctttttccgcgtgtcggaggagcagcagaagcagcagcgcagggagatgctgaaggacccttttgtgcgctccaagctcatctcgccgcctaccaacttcaaccacctagtacacgtgggccctgccaacgggcggcccggcgccagggacaagtccccggctcccgaagagaagggccgagttgcccgcggctccggcccacagcggccccacagcttctccgaggcgttgcggcgcccagcctccatgggcagcgaaggcctcggtggagacgcagaccccatgaagaggaaaccctggacatccctgtccagcgagtctgtgtcctgcccccagggatcgctgagccctgcaacctccctaatgcaggtctcagaacggccccgaagcctccccctatcccctgaattggagagctctccttgatgccctctgttagggcccacccca

SEQ ID NO: 6 - protein, 1551 aa, Homo sapiens
MERRLRALEQLARGEAGGCPGLDGLLDLLLALHHELSSGPLRRERSVAQFLSWASPFVSKVKELRLQRDDFEILKVIGRGAFGEVTVVRQRDTGQIFAMKMLHKWEMLKRAETACFREERDVLVKGDSRWVTTLHYAFQDEEYLYLVMDYYAGGDLLTLLSRFEDRLPPELAQFYLAEMVLAIHSLHQLGYVHRDVKPDNVLLDVNGHIRLADFGSCLRLNTNGMVDSSVAVGTPDYISPEILQAMEEGKGHYGPQCDWWSLGVCAYELLFGETPFYAESLVETYGKIMNHEDHLQFPPDVPDVPASAQDLIRQLLCRQEERLGRGGLDDFRNHPFFEGVDWERLASSTAPYIPELRGPMDTSNFDVDDDTLNHPGTLPPPSHGAFSGHHLPFVGFTYTSGSHSPESSSEAWAALERKLQCLEQEKVELSRKHQEALHAPTDHRELEQLRKEVQTLRDRLPEMLRDKASLSQTDGPPAGSPGQDSDLRQELDRLHRELAEGRAGLQAQEQELCRAQGQQEELLQRLQEAQEREAATASQTRALSSQLEEARAAQRELEAQVSSLSRQVTQLQGQWEQRLEESSQAKTIHTASETNGMGPPEGGPQEAQLRKEVAALREQLEQAHSHRRSGKEEALCQLQEENRRLSREQERLEAELAQEQESKQRLEGERRETESNWEAQLADILSWVNDEKVSRGYLQALATKMAEELESLRNVGTQTLPARPLDHQWKARRLQKMEASARLELQSALEAEIRAKQGLQERLTQVQEAQLQAERRLQEAEKQSQALQQELAMLREELRARGPVDTKPSNSLIPFLSFRSSEKDSAKDPGISGEATRHGGEPDLRPEGRRSLRMGAVFPRAPTANTASTEGLPAKPGSHTLRPRSFPSPTKCLRCTSLMLGLGRQGLGCDACGYFCHTTCAPQAPPCPVPPDLLRTALGVHPETGTGTAYEGFLSVPRPSGVRRGWQRVFAALSDSRLLLFDAPDLRLSPPSGALLQVLDLRDPQFSATPVLASDVIHAQSRDLPRIFRVTTSQLAVPPTTCTVLLLAESEGERERWLQVLGELQRLLLDARPRPRPVYTLKEAYDNGLPLLPHTLCAAILDQDRLALGTEEGLFVIHLRSNDIFQVGECRRVQQLTLSPSAGLLVVLCGRGPSVRLFALAELENIEVAGAKIPESRGCQVLAAGSILQARTPVLCVAVKRQVLCYQLGPGPGPWQRRIRELQAPATVQSLGLLGDRLCVGAAGGFALYPLLNEAAPLALGAGLVPEELPPSRGGLGEALGAVELSLSEFLLLFTTAGIYVDGAGRKSRGHELLWPAAPMGWGYAAPYLTVFSENSIDVFDVRRAEWVQTVPLKKVRPLNPEGSLFLYGTEKVRLTYLRNQLAEKDEFDIPDLTDNSRRQLFRTKSKRRFFFRVSEEQQKQQRREMLKDPFVRSKLISPPTNFNHLVHVGPANGRPGARDKSPAPEEKGRVARGSGPQRPHSFSEALRRPASMGSEGLGGDADPMKRKPWTSLSSESVSCPQGSLSPATSLMQVSERPRSLPLSPELESSP